(19) United States
(12) Patent Application Publication
(10) Pub. No.: US 2021/0046120 A1
(43) Pub. Date: Feb. 18, 2021

(54) THROMBOSOMES AS AN ANTICOAGULANT REVERSAL AGENT

(71) Applicant: Cellphire, Inc., Rockville, MD (US)

(72) Inventors: Keith Andrew Moskowitz, Westfield, IN (US); Braden Carl Ishler, Gaithersburg, SD (US); William Matthew Dickerson, Washington, DC (US); Amber Nicole Lee, Rockville, MD (US); Shan Xu, Rockville, MD (US)

(21) Appl. No.: 16/994,330

(22) Filed: Aug. 14, 2020

Related U.S. Application Data
(60) Provisional application No. 62/887,985, filed on Aug. 16, 2019.
(60) Provisional application No. 63/065,337, filed on Aug. 13, 2020.

(51) Int. Cl. A61K 35/19; A61K 31/352; A61P 7/04; A61K 31/7008; A61K 47/42; A61K 47/26

(52) U.S. Cl. CPC A61K 35/19; A61K 31/352; A61K 47/26; A61K 31/7008; A61K 47/42; A61P 7/04

(57) ABSTRACT

In some embodiments provided herein is a method of treating a coagulopathy in a subject, the method including administering to the subject in need thereof an effective amount of a composition including platelets or platelet derivatives and an incubating agent including one or more salts, a buffer, optionally a cryoprotectant, and optionally an organic solvent.

### CROSS-REFERENCE TO RELATED APPLICATIONS

This application claims priority to U.S. Provisional Application Ser. No. 62/887,985, filed on Aug. 16, 2019 and U.S. Provisional Application Ser. No. 63/065,337, filed on Aug. 13, 2020, each of which is incorporated herein by reference in its entirety.

### FIELD OF THE INVENTION

This disclosure serves to describe the use of thrombosomes as a treatment for drug-induced coagulopathy. Anticoagulant drugs such as warfarin, heparin, and the NOAC class inhibit various plasma factors of the coagulation cascade, resulting in increased bleeding potential. Here we demonstrate that thrombosomes circumvent or overcome this inhibition to restore hemostasis.

### BACKGROUND

Anticoagulant drugs are common in the U.S. adult population and employ a wide variety of mechanisms to disable segments of the clotting cascade. Anticoagulants are used to treat a number of cardiac or thromboembolic events. For example, warfarin (e.g., COUMADIN®) is approved for the prophylaxis and treatment of venous thrombosis and its extension, pulmonary embolism; the prophylaxis and treatment of thromboembolic complications associated with atrial fibrillation and/or cardiac valve replacement; the reduction in the risk of death, recurrent myocardial infarction, and thromboembolic events such as stroke or systemic embolization after myocardial infarction (see, e.g., Prescribing Information for warfarin (COUMADIN®)). As another example, heparin is approved for the treatment of thrombophlebitis, phlebothrombosis, and cerebral, coronary, and retinal vessel thrombosis to prevent extension of clots and thromboembolic phenomena. It is also used prophylactically to prevent the occurrence of thromboembolism, and to prevent clotting during dialysis and surgical procedures, particularly vascular surgery. Other drugs that have anticoagulant properties can include agents that inhibit factor IIa (thrombin) (also called anti-IIa agents, thrombin inhibitors, or direct thrombin inhibitors, depending on the mechanism of action), including dabigatran (e.g., PRADAXA®), argatroban, and hirudin; and agents that inhibit factor Xa, including rivaroxaban (e.g., XARELTO®), apixaban (e.g., ELIQUIS®), edoxaban (e.g., SAVAYSA®), and fondaparinux (e.g., ARIXTRA®). Traditional anticoagulants can include warfarin (e.g., COUMADIN®) and heparin/LMWH (low molecular weight heparins). Additional anticoagulants include heparainoids, factor IX inhibitors, Factor XI inhibitors, Factor VIIa inhibitors, and Tissue Factor inhibitors.

Anticoagulants, however, are responsible for many adverse drug-related events (ADEs) annually, including 10% of all inpatient ADEs, an estimated up to 34,000 ADEs per year in nursing homes. Warfarin has been implicated in 17% of all emergency hospital visits in adults >65 years. At least 2000 patients suffer fatal bleeding after vitamin K-antagonist therapy with warfarin.

Warfarin reversal therapies can also be very expensive, with the exception of vitamin K—which may be no less dangerous than warfarin. For example, Kcentra (Prothrombin complex concentrate; PCC) costs about $5100/dose.

NOACs have similar bleeding risk to coumadin, cannot be monitored and present a challenge for reversal situations when emergency surgery is required.

Overdose and adverse events related to these drugs carry the risk of serious bleeding and related complications in the patient population. There is therefore a need in the art for the treatment of coagulopathy, such as anticoagulant-induced coagulopathy.

### SUMMARY OF THE INVENTION

Provided herein in some embodiments is a method of treating a coagulopathy in a subject, the method including administering to the subject in need thereof an effective amount of a composition including platelets or platelet derivatives and an incubating agent including one or more salts, a buffer, optionally a cryoprotectant, and optionally an organic solvent.

In some embodiments, provided herein is a method of treating a coagulopathy in a subject, the method including administering to the subject in need thereof an effective amount of a composition prepared by a process including incubating platelets with an incubating agent including one or more salts, a buffer, optionally a cryoprotectant, and optionally an organic solvent, to form the composition.

In some embodiments, provided herein is a method of restoring normal hemostasis in a subject, the method including administering to the subject in need thereof an effective amount of a composition including platelets or platelet derivatives and an incubating agent including one or more salts, a buffer, optionally a cryoprotectant, and optionally an organic solvent.

In some embodiments, provided herein is a method of restoring normal hemostasis in a subject, the method including administering to the subject in need thereof an effective amount of a composition prepared by a process including incubating platelets with an incubating agent including one or more salts, a buffer, optionally a cryoprotectant, and optionally an organic solvent, to form the composition.

In some embodiments, provided herein is a method of preparing a subject for surgery, the method including administering to the subject in need thereof an effective amount of a composition including platelets or platelet derivatives and an incubating agent including one or more salts, a buffer, optionally a cryoprotectant, and optionally an organic solvent. Implementations can include one or more of the following features. The surgery can be an emergency surgery. The surgery can be a scheduled surgery.

In some embodiments, provided herein is a method of preparing a subject for surgery, the method including administering to the subject in need thereof an effective amount of a composition prepared by a process including incubating platelets with an incubating agent including one or more salts, a buffer, optionally a cryoprotectant, and optionally an organic solvent, to form the composition. Implementations can include one or more of the following features. The surgery can be an emergency surgery. The surgery can be a scheduled surgery.

In some implementations of the above methods, the subject has been treated or is being treated with an anticoagulant. In some embodiments, treatment with the anticoagulant can be stopped. In some embodiments, treatment with the anticoagulant can be continued.

In some embodiments, provided herein is a method of ameliorating the effects of an anticoagulant in a subject, the method including administering to the subject in need thereof an effective amount of a composition including platelets or platelet derivatives and an incubating agent including one or more salts, a buffer, optionally a cryoprotectant, and optionally an organic solvent.

In some embodiments, provided herein is a method of ameliorating the effects of an anticoagulant in a subject, the method including administering to the subject in need thereof an effective amount of a composition prepared by a process including incubating platelets with an incubating agent including one or more salts, a buffer, optionally a cryoprotectant, and optionally an organic solvent, to form the composition.

In some embodiments, the effects of the anticoagulant can be the result of an overdose of the anticoagulant.

In some embodiments, the anticoagulant can be selected from the group consisting of dabigatran, argatroban, hirudin, rivaroxaban, apixaban, edoxaban, fondaparinux, warfarin, heparin, a low molecular weight heparin, and a supplement. In some embodiments, the anticoagulant can be warfarin. In some embodiments, the anticoagulant can be heparin.

In some embodiments of any of the methods herein, before the administering, the subject can have an INR of at least 4.0. In some embodiments, after the administering, the subject can have an INR of 3.0 or less. In some embodiments, after the administering, the subject can have an INR of 2.0 or less.

In some embodiments of any of the methods herein, before the administering, the subject can have an INR of at least 3.0. In some embodiments, after the administering, the subject can have an INR of 2.0 or less.

Some embodiments of any of the methods herein can include one or more of the following features. Administering can include administering topically. Administering can include administering parenterally. Administering can include administering intravenously. Administering can include administering intramuscularly. Administering can include administering intrathecally. Administering can include administering subcutaneously. Administering can include administering intraperitoneally. The composition can be dried prior to the administration step. The composition can be rehydrated following the drying step. The composition can be freeze-dried prior to the administration step. The composition can be rehydrated following the freeze-drying step. The incubating agent can include one or more salts selected from phosphate salts, sodium salts, potassium salts, calcium salts, magnesium salts, and a combination of two or more thereof. The incubating agent can include a carrier protein. The buffer can include HEPES, sodium bicarbonate (NaHCO₃), or a combination thereof. The composition can include one or more saccharides. The one or more saccharides can include trehalose. The one or more saccharides can include polysucrose. The one or more saccharides can include dextrose. The composition can include an organic solvent. The platelets or platelet derivatives can include thrombosomes.

### BRIEF DESCRIPTION OF THE DRAWINGS

FIG. 1 shows peak thrombin generation obtained by adding 400×10³/μL thrombosomes to warfarin plasma at various INR levels.

FIG. 2 shows endogenous thrombin potential (ETP) values obtained by adding 400×10³/μL thrombosomes to plasma at various INR levels.

FIG. 3 shows peak thrombin generation by thrombosomes and by fresh platelets in INR 2 warfarin plasma.

FIG. 4 shows the effect on r-time of warfarin plasma samples in a TEG assay as a result of the addition of 300×10³/μL thrombosomes.

FIG. 5 shows that thrombosomes provide a dose-dependent increase in peak thrombin generation. These data were collected in the background of whole blood with an endogenous platelet count of 150×10³/μL.

FIG. 6 shows a plot of the concentration of platelets or thrombosomes versus peak thrombin generation.

FIG. 7A shows a plot of the concentration of platelets, thrombosomes, or a combination thereof versus peak thrombin generation in INR-2 plasma.

FIG. 7B shows thrombin generation in INR-1 plasma, INR-2 plasma (treated with warfarin), and INR-2 plasma (treated with warfarin) plus thrombosomes (150×10³/μL), for four different batches of thrombosomes.

FIG. 8 shows the generation of thrombus by thrombosomes in warfarin plasma in a shear-dependent collagen adhesion assay under flow (T-TAS®)

FIG. 9 shows a plot of the time to generation of thrombus increasing with increasing concentrations of rivaroxaban in whole blood (WB).

FIG. 10A shows a plot of the time to generation of thrombus in the presence of 3 μM rivaroxaban decreasing with the addition of thrombosomes.

FIG. 10B shows a plot of the time to generation of thrombus in control plasma, in plasma treated with 3 μM rivaroxaban, and in plasma treated with 3 μM rivaroxaban and 300×10³/μL thrombosomes.

FIG. 10C shows a plot of the time to generation of occlusion of T-TAS® AR chip from FIG. 10B.

FIG. 11A shows the effect of thrombosomes in warfarin plasma (INR=1.6) compared to standard plasma (INR=1.0), measured in terms of R-time (start of clot formation).

FIG. 11B shows the effect of thrombosomes in warfarin plasma (INR=1.6) compared to standard plasma (INR=1.0), measured in terms of R-time, plotted on a log-scale x-axis.

FIG. 12A shows the effect of thrombosomes in warfarin plasma (INR=1.6) in terms of alpha angle (also called angle).

FIG. 12B shows the effect of thrombosomes in warfarin plasma (INR=1.6) in terms of alpha angle (also called angle), plotted on a log-scale x-axis.

FIG. 13 shows the effect of thrombosomes in warfarin plasma (INR=1.6) in terms of maximum amplitude (MA).

FIG. 14 shows the effect of thrombosomes in warfarin plasma (INR=1.6) in terms of maximum amplitude (MA), plotted on a log-scale x-axis.

FIG. 15 shows a plot of the decrease in lag time for samples with different INR values supplemented thrombosomes.

FIG. 16 is an exemplary thrombelastography (TEG) waveform with parameters labeled.

FIG. 17 is a plot of R-time for various INR values of warfarin plasma, with or without supplementation with various concentrations of thrombosomes.

FIG. 18 is a plot of activated clotting time in plasma levels of various INR levels, with and without supplemented thrombosomes.

FIG. 19 shows the effect of thrombosomes on whole blood (normal; INR=2; INR=3; and INR=6.2)

FIG. 20A shows the effect on peak thrombin generation of thrombosomes in plasma with INRs of 1 and 2.

FIG. 20B shows the effect on peak thrombin generation of thrombosomes in plasma with an INR of 3.

FIG. 20C shows the effect on peak thrombin generation of thrombosomes in plasma with INRs of 1 and 6.

FIG. 21A shows the effect on endogenous thrombin potential of thrombosomes in plasma with INRs of 1 and 2.

FIG. 21B shows the effect on endogenous thrombin potential of thrombosomes in plasma with an INR of 3.

FIG. 21C shows the effect on endogenous thrombin potential of thrombosomes in plasma with INRs of 1 and 6.

FIG. 22A shows the effect on peak thrombin generation of thrombosomes in plasma with INRs of 1, 2, 3, and 6 (left) and a zoomed-in image of the same data from 0 to 30 nM (right) for a replicate of thrombosomes batch 1.

FIG. 22B shows the effect on peak thrombin generation of thrombosomes in plasma with INRs of 1, 2, 3, and 6 for a replicate of thrombosomes batch 1.

FIG. 22C shows the effect on peak thrombin generation of thrombosomes in plasma with INRs of 1, 2, 3, and 6 for a replicate of thrombosomes batch 1.

FIG. 22D shows the effect on peak thrombin generation of thrombosomes in plasma with INRs of 1, 2, 3, and 6 (left) and a zoomed-in image of the same data from 0 to 2.5 nM (right) for thrombosomes batch 2.

FIG. 22E shows the effect on peak thrombin generation of thrombosomes in plasma with INRs of 1, 2, and 3 for thrombosomes batch 3.

FIG. 23A shows aPTT values for plasma and plasma treated with heparin.

FIG. 23B shows thrombin generation for plasma treated with heparin, with the addition of fresh platelets or thrombosomes initiated with PPP low reagent.

FIG. 23C shows thrombin generation for plasma treated with heparin, with the addition of fresh platelets or thrombosomes initiated with PRP reagent.

FIG. 24A shows aPTT values for plasma, plasma treated with heparin, and plasma treated with heparin and protamine sulfate.

FIG. 24B shows thrombin generation for plasma treated with heparin and thrombosomes, without (relatively flat lines) or with (curves) addition of protamine sulfate, initiated with PPP low reagent.

FIG. 24C shows thrombin generation for plasma treated with heparin and thrombosomes, without (relatively flat lines) or with (curves) addition of protamine sulfate, initiated with PRP reagent.

FIG. 25A shows thrombin generation for control plasma, plasma treated with dabigatran, or plasma treated with dabigatran and thrombosomes initiated with PRP reagent.

FIG. 25B shows the time to peak (TTP) in a thrombin generation assay for control plasma, plasma treated with dabigatran, or plasma treated with dabigatran and thrombosomes initiated with PRP reagent.

### DETAILED DESCRIPTION

Before embodiments of the present invention are described in detail, it is to be understood that the terminology used herein is for the purpose of describing particular embodiments only, and is not intended to be limiting. Unless defined otherwise, all technical and scientific terms used herein have the same meaning as commonly understood by one of ordinary skill in the art to which the term belongs. Although any methods and materials similar or equivalent to those described herein can be used in the practice of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a saccharide” includes reference to one or more saccharides, and equivalents thereof known to those skilled in the art. Furthermore, the use of terms that can be described using equivalent terms include the use of those equivalent terms. Thus, for example, the use of the term “subject” is to be understood to include the terms “patient”, “person”, “animal”, “human”, and other terms used in the art to indicate one who is subject to a medical treatment. The use of multiple terms to encompass a single concept is not to be construed as limiting the concept to only those terms used.

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

As used herein and in the appended claims, the term “platelet” can include whole platelets, fragmented platelets, platelet derivatives, or thrombosomes. “Platelets” within the above definition may include, for example, platelets in whole blood, platelets in plasma, platelets in buffer optionally supplemented with select plasma proteins, cold stored platelets, dried platelets, cryopreserved platelets, thawed cryopreserved platelets, rehydrated dried platelets, rehydrated cryopreserved platelets, lyopreserved platelets, thawed lyopreserved platelets, or rehydrated lyopreserved platelets. “Platelets” may be “platelets” of mammals, such as of humans, or such as of non-human mammals.

As used herein, “thrombosomes” (sometimes also herein called “Tsomes” or “Ts”, particularly in the Examples and Figures) are platelet derivatives that have been treated with an incubating agent (e.g., any of the incubating agents described herein) and lyopreserved (such as freeze-dried). In some cases, thrombosomes can be prepared from pooled platelets. Thrombosomes can have a shelf life of 2-3 years in dry form at ambient temperature and can be rehydrated with sterile water within minutes for immediate infusion. One example of thrombosomes are THROMBOSOMES, which are in clinical trials for the treatment of acute hemorrhage in thrombocytopenic patients. Agents that inhibit Factor IIa, VIIa, IX, Xa, XI, Tissue Factor, or vitamin K-dependent synthesis of clotting factors (e.g., Factor II, VII, IX, or X) or that activate antithrombin (e.g., antithrombin III) are anticoagulants for the purpose of the present disclosure. Other mechanisms of anticoagulants are known. Non-limiting examples of anticoagulants include dabigatran, argatroban, hirudin, rivaroxaban, apixaban, edoxaban, fondaparinux, warfarin, heparin, and low molecular weight heparins (e.g., dalteparin, enoxaparin, tinzaparin, ardeparin, nadroparin, reveparin, danaparoid). Additional non-limiting examples of anticoagulants include tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, and fluindione. In some embodiments, the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, and fluindione.

As used herein, an “anticoagulant” is an antithrombotic that does not include antiplatelet agents. Examples of antiplatelet agents include aspirin, cangrelor, ticagrelor, clopidogrel (e.g., PLAVIX®), prasugrel eptifibatide (e.g., INTEGRILIN®), tirofiban (e.g., AGGRASTAT®), and abciximab (e.g., REOPRO®). Typically, agents that inhibit P2Y receptors (e.g., P2Y12), glycoprotein IIb/IIIa, or that antagonize thromboxane synthase or thromboxane receptors, are considered to be antiplatelet agents. Other mechanisms of antiplatelet agents are known. As used herein, aspirin is considered to be an antiplatelet agent but not an anticoagulant.

Overcoming the effect of an anticoagulant varies according to the anticoagulant drug pharmacological action. In the case of advanced notice, as in a pre-planned surgery, the anti-coagulant dose can sometimes be tailored back before the surgery; however, there may be cases where such a reduction in dose is not advisable. In the case where an anti-coagulant need reversing quickly (e.g., for emergency surgery), reversal agents are typically slow acting, expensive, or carry significant risk to the patient. Below are some non-limiting examples of reversal agents for marketed anti-coagulants.

Warfarin (e.g., COUMADIN®)—Warfarin works to prevent the activity of vitamin K in the liver which is a necessary co-factor to produce multiple coagulation factors. Warfarin reversal can sometimes be done be by dosing vitamin K or prothrombin complex concentrate (PCC). Vitamin K is low-cost and slow acting (more than 24 hrs PO) but can pose significant risk of inducing thrombosis in the patient, while PCC is expensive at roughly $5000/dose.

Dabigatran (e.g., PRADAXA®)—Dabigatran is a direct inhibitor of thrombin. The monoclonal antibody therapy idarucizumab (e.g., PRAXBIND®, Boehringer-Ingelheim, Germany) at dose of 5 grams (at two dose intervals each 2.5 grams) can typically reverse the effects of dabigatran within a few minutes. One wholesale price is $3482.50 for such a treatment.

Rivaroxaban (e.g., XARELTO®)—Rivaroxaban is a direct Factor Xa inhibitor. Rivaroxaban is reversed by Andexanet Alfa (e.g., ANDEXXA®), a recombinant Factor Xa decoy. This treatment can cost roughly $50,000 for a high-dose treatment.

Apixaban (e.g., ELIQUIS®)—Apixaban is a direct Factor Xa inhibitor. Apixaban is reversed by Andexanet Alfa, a recombinant Factor Xa decoy. This treatment costs roughly can cost $50,000 for a high-dose treatment.

Edoxaban (e.g., SAVAYSA®, LIXIANA®)—Edoxaban is a direct Factor Xa inhibitor. Exoxaban does not have an approved reversal agent. Ciraparantag (aripazine) and Andexanet Alfa have not been clinically proven to be appropriate.

Heparin and low molecular weight heparins are activators of antithrombin III (AT). AT inactivates proteases such as thrombin and Factor Xa. Protamine sulfate is a highly positively-charged polypeptide that binds to the negatively charged heparin and prevents its action on AT. Protamine sulfate is typically dosed at about 1.0 to about 1.5 mg/100 IU of active heparin.

Platelet-derived products are not currently used as a treatment method for anticoagulant drugs.

Treatments for anticoagulant drugs are not necessarily targeted antidotes. Some novel anticoagulant treatments, such as Andexanet Alfa (e.g., ANDEXXA®), have seen some success, yet can be expensive. As such, emergency treatments (pre-op, trauma, and the like) are typically blanket precautions to avoid or mitigate hemorrhage. Non-limiting examples include infusion of plasma, red blood cells, and anti-fibrinolytics. Platelet derivatives (e.g., lyopreserved platelets (e.g., thrombosomes)) may be an effective alternative or supplement to these general treatments.

Without being bound by any particular theory, it is believed that thrombosomes can work at least in part by providing a procoagulant negatively charged surface to augment thrombin generation above and beyond that suppressed by the anti-coagulants.

Products and methods are described herein for controlling bleeding and improving healing. The products and methods described herein can also be used to counteract the activity of an anticoagulant (e.g., warfarin (e.g., COUMADIN®), heparin, LMWH, dabigatran (e.g., PRADAXA®), argatroban, hirudin, rivaroxaban (e.g., XARELTO®), apixaban (e.g., ELIQUIS®), edoxaban (e.g., SAVAYSA®), fondaparinux (e.g., ARIXTRA®). The products and methods described herein are directed toward embodiments that aid in the closure and healing of wounds.

In certain embodiments, a composition comprising platelets such as lyophilized platelets or platelet derivatives may be delivered to a wound on the surface of or in the interior of a patient. In various embodiments, a composition comprising platelets or platelet derivatives can be applied in selected forms including, but not limited to, adhesive bandages, compression bandages, liquid solutions, aerosols, matrix compositions, and coated sutures or other medical closures. In embodiments, a platelet derivative may be administered to all or only a portion of an affected area on the surface of a patient. In other embodiments, a composition comprising platelets such as lyophilized platelets or platelet derivatives may be administered systemically, for example via the blood stream. In embodiments, an application of the platelet derivative can produce hemostatic effects for 2 or 3 days, preferably 5 to 10 days, or most preferably for up to 14 days.

Some embodiments provide a method of treating a coagulopathy in a subject, the method comprising administering to the subject in need thereof an effective amount of a composition comprising platelets such as lyophilized platelets or platelet derivatives and an incubating agent comprising one or more salts, a buffer, optionally a cryoprotectant (also called a lyophilizing agent), and optionally an organic solvent.

Some embodiments provide a method of treating a coagulopathy in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments of any of the methods described herein, the coagulopathy is the result of an anticoagulant.

Some embodiments provide a method of treating coagulopathy in a subject, wherein the subject has been treated or is being treated with an anticoagulant, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of treating coagulopathy in a subject, wherein the subject has been treated or is being treated with an anticoagulant, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some embodiments provide a method of restoring normal hemostasis in a subject, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of restoring normal hemostasis in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some embodiments provide a method of restoring normal hemostasis in a subject, wherein the subject has been treated or is being treated with an anticoagulant, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of restoring normal hemostasis in a subject, wherein the subject has been treated or is being treated with an anticoagulant, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Compositions as described herein can also be administered to prepare a subject for surgery, in some cases. For some patients taking an anticoagulant, it may be difficult or impossible to reduce the dosage of the anticoagulant before surgery (e.g., in the case of trauma or other emergency surgery). For some patients taking an anticoagulant, it may be inadvisable to reduce the dosage of the anticoagulant before surgery (e.g., if the patient would be at risk of a thrombotic event (e.g., deep vein thrombosis, pulmonary embolism, or stroke) if the dosage of the anticoagulant were reduced over time.

Accordingly, some embodiments provide a method of preparing a subject for surgery, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of preparing a subject for surgery, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some embodiments provide a method of preparing a subject for surgery, wherein the subject has been treated or is being treated with an anticoagulant, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of preparing a subject for surgery, wherein the subject has been treated or is being treated with an anticoagulant, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments, a surgery can be an emergency surgery (e.g., in the case of trauma) or a scheduled surgery.

In some embodiments of any of the methods described herein, treatment with an anticoagulant can be stopped (e.g., in preparation for surgery). In some embodiments, treatment with an anticoagulant can continue.

In some embodiments of any of the methods described herein, the subject may or may not be also treated with an anticoagulant reversal agent (e.g., idarucizumab, Andexanet Alfa, Ciraparantag (aripazine), protamine sulfate, vitamin K). In some embodiments, the subject is not also treated with an anticoagulant reversal agent. In some embodiments, the subject is also treated with an anticoagulant reversal agent. It will be understood that an anticoagulant reversal agent can be chosen based on the anticoagulant administered to the subject.

Some embodiments provide a method of ameliorating the effects of an anticoagulant in a subject, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of ameliorating the effects of an anticoagulant in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some cases, the effects of an anticoagulant may need to be ameliorated due to an incorrect dosage of an anticoagulant. For example, in some embodiments, the effects of an anticoagulant can be ameliorated following an overdose of the anticoagulant. In some cases, the effects of an anticoagulant may need to be ameliorated due to a potential for interaction with another drug (e.g., a second anticoagulant). For example, in some embodiments, the effects of an anticoagulant can be ameliorated following an erroneous dosing of two or more drugs, at least one of which is an anticoagulant.

In some embodiments of any of the methods described herein, the composition can further comprise an active agent, such as an anti-fibrinolytic agent. Non-limiting examples of anti-fibrinolytic agents include F-aminocaproic acid (EACA), tranexamic acid, aprotinin, aminomethylbenzoic acid, and fibrinogen. In some embodiments, platelets or platelet derivatives can be loaded with an active agent, such as an anti-fibrinolytic agent.

Clotting parameters of blood (e.g., the subject's blood) can be assessed at any appropriate time during the methods described herein. For example, one or more clotting parameters of blood can be assessed before administration of a composition comprising platelets such as lyophilized platelets or platelet derivatives as described herein, e.g., in order to determine the need for administration of a composition comprising platelets or platelet derivatives as described herein. As another example, one or more clotting parameters of blood can be assessed after administration of a composition comprising platelets such as lyophilized platelets or platelet derivatives as described herein, e.g., in order to determine the effectiveness of the administered composition, to determine whether additional administration of the composition is warranted, or to determine whether it is safe to perform a surgical procedure.

Accordingly, any of the methods described herein can include steps of assessing one or more clotting parameters of blood before administration of a composition comprising platelets or platelet derivatives as described herein, assessing one or more clotting parameters of blood after administration of a composition comprising platelets such as lyophilized platelets or platelet derivatives as described herein, or both.

Any appropriate method can be used to assess clotting parameters of blood. Non-limiting examples of methods include the prothrombin time assay, international normalized ratio (INR), thrombin generation (TGA; which can be used to generate parameters such as, e.g., peak thrombin, endogenous thrombin potential (ETP), and lag time), thromboelastography (TEG), activated clotting time (ACT), and partial thromboplastin time (PTT or aPTT).

INR is a standard method of determining dosing, see equation below, where “PT(x)” is the result of the prothrombin time assay, while the ISI constant is dependent on the manufacturer of the Tissue Factor used in the prothrombin time assay.

$$INR = {( \frac{PT ( patient )}{PT ( normal )} )}^{ISI constant}$$

Warfarin inhibits the synthesis of four major plasma proteins that are integral to healthy clot formation. A therapeutic maintenance dose of warfarin is typically targeted to an INR of about 2.0 to about 3.0. Thrombosomes present a unique treatment to restore hemostasis in the presence of warfarin-type drugs. Warfarin dose can be expressed by INR, a ratio that increases with the amount of warfarin (1 is a normal value).

In some embodiments, a subject has an INR of more than 2.0 (e.g., at least 2.2, at least 2.4, at least 2.5, at least 2.6, at least 2.8, at least 3.0, at least 3.2, at least 3.4, at least 3.5, at least 3.6, at least 3.8, at least 4.0, at least 4.2, at least 4.4, at least 4.5, at least 4.6, at least 4.8, or at least 5.0) before administration of a composition comprising platelets such as lyophilized platelets or platelet derivatives as described herein. In some embodiments, a subject (e.g., a subject being treated with an anticoagulant, such as warfarin) has an INR of from 2.0 to 3.0, such as from 2.2 to 2.8, such as from 2.4 to 2.6, such as 2.5.

In some embodiments, a subject has a lower INR (or a normal INR) after administration of a composition comprising platelets such as lyophilized platelets or platelet derivatives as described herein. For example, a subject can have an INR of 3.0 or less (e.g., less than 2.8, less than 2.6, less than 2.5, less than 2.4, less than 2.2, less than 2.0, less than 1.8, less than 1.6, less than 1.5, less than 1.4, less than 1.2, or less than 1.0) after administration of a composition comprising platelets or platelet derivatives ad described herein.

Thrombin Generation

The thrombin generation assay measured the production of thrombin after sample activation via a pro-coagulation agent resulting of thrombin enzymatic cleavage of a fluorescent peptide and release of fluorescent molecule. The peak thrombin is a measure of the maximum thrombin produced, lag time, the time to start of thrombin production, and ETP as the total thrombin potentially produced.

In some embodiments, a patient can have a peak thrombin of about 60 nM to about 170 nM, such as about 65 nM to about 170 nM, such as about 65 nM to about 120 nM, such as about 80 nM, before administration of a composition comprising platelets or platelet derivatives as described herein.

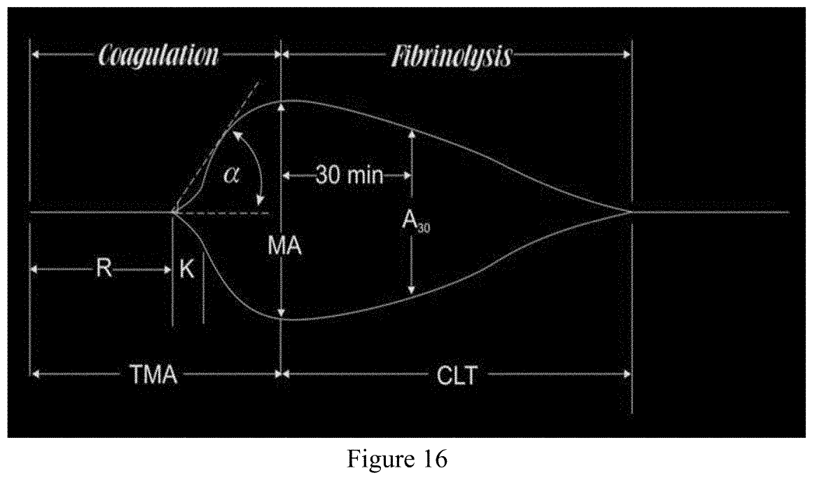

TEG assesses intrinsic hemostasis via plots of clot strength over time. Calcium chloride (CaCl₂)) is typically used as the initiating reagent. A TEG waveform (see, e.g., FIG. 16) has multiple parameters that can provide information about clotting.

R-time=reaction time (s)—time of latency from start of test to initial fibrin formation.

K=kinetics (s)—speed of initial fibrin formation, time taken to achieve a certain level of clot strength (e.g., an amplitude of 20 mm)

alpha angle=slope of line between R and K—measures the rate of clot formation.

MA=maximum amplitude (mm)—represents the ultimate strength of the fibrin clot.

A₃₀=amplitude 30 minutes after maximum amplitude is reached—represents rate of lysis phase.

In hypocoagulable blood states, R-time increases and MA decreases. R-time typically provides a broader response range than MA.

In the Total Thrombus-formation Analysis System (T-TAS®, FUJIMORI KOGYO CO., LTD), the sample is forced through collagen-coated microchannels using mineral oil. Changes in pressure are used to assess thrombus formation. The Occlusion Start Time is time it takes to reach 10 kPa, and the Occlusion Time=time it takes to each Δ80 kPa using an AR chip (e.g., Zacros Item No, TC0101). According to the manufacturer, an AR chip can be used for analyzing the formation of a mixed white thrombus consisting chiefly of fibrin and activated platelets. It has a flow path (300 μm wide by 50 μm high) coated with collagen and tissue factors and can be used to analyze the clotting function and platelet function. In comparison, a PL chip can be used for analyzing the formation of a platelet thrombus consisting chiefly of activated platelets. A PL chip has a flow path coated with collagen only and can be used to analyze the platelet function.

The ACT assay is the most basic, but possibly most reliable, way to measure clotting time (tACT), determined by a magnet's resistance to gravity as a clot forms around it. Typical donor blood has a tACT˜200-300 s using only CaCl₂.

Some embodiments provide a method of increasing thrombin generation in a subject, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments, provide a method of increasing thrombin generation in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Some embodiments provide a method of increasing peak thrombin in a subject, the method comprising administering to the subject in need thereof an effective amount of a composition comprising platelets such as lyophilized platelets or platelet derivatives and an incubating agent comprising one or more salts, a buffer, optionally a cryoprotectant, and optionally an organic solvent.

Some embodiments provide a method of increasing peak thrombin in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

In some embodiments, prior to the administering, the peak thrombin of the subject was below 66 nM (e.g., below 64 nM, 62 nM, 60 nM, 55 nM, 50 nM, 45 nM, 40 nM, 35 nM, 30 nM, 25 nM, 20 nM, 15 nM, 10 nM, or 5 nM). In some embodiments, after the administering, the peak thrombin of the subject is above 66 nM (e.g., above 68 nM, 70 nM, 75 nM, 80 nM, 85 nM, 90 nM, 95 nM, 100 nM, 110 nM, 120 nM, 130 nM, 140 nM, or 150 nM). In some embodiments, after the administering, the peak thrombin of the subject is between 66 and 166 nM. Peak thrombin can be measured by any appropriate method.

An “effective amount” as used herein is an amount of the composition that comprises an amount of platelets such as lyophilized platelets or platelet derivatives (e.g., thrombosomes) effective in treating the subject. Such an amount of platelets or platelet derivatives (e.g., thrombosomes) includes any appropriate dosage of a composition comprising platelets or platelet derivatives as described herein that can be administered to the subject. For example, in some embodiments, a dose of a composition comprising platelets or platelet derivatives (e.g., thrombosomes) can include about 1.0×10⁷ particles to about 1.0×10¹⁰ particles, such as about 1.6×10⁷ particles (e.g., thrombosomes)/kg to about 1.0×10¹⁰ particles/kg (e.g., about 1.6×10⁷ to about 5.1×10⁹ particles/kg, about 1.6×10⁷ to about 3.0×10⁹ particles/kg, about 1.6×10⁷ to about 1.0×10⁹ particles/kg, about 1.6×10⁷ to about 5.0×10⁸ particles/kg, about 1.6×10⁷ to about 1.0×10⁸ particles/kg, about 1.6×10⁷ to about 5.0×10⁷ particles/kg, about 5.0×10⁷ to about 1.0×10⁸ particles/kg, about 1.0×10⁸ to about 5.0×10⁸ particles/kg, about 5.0×10⁸ to about 1.0×10⁹ particles/kg, about 1.0×10⁹ to about 5.0×10⁹ particles/kg, or about 5.0×10⁹ to about 1.0×10¹⁰ particles/kg).

In some embodiments of the methods herein, the composition is administered topically. In some embodiments, topical administration can include administration via a solution, cream, gel, suspension, putty, particulates, or powder. In some embodiments, topical administration can include administration via a bandage (e.g. an adhesive bandage or a compression bandage) or medical closure (e.g., sutures, staples)); for example the platelet derivatives (e.g., lyopreserved platelets (e.g., thrombosomes)) can be embedded therein or coated thereupon), as described in PCT Publication No. W2017/040238 (e.g., paragraphs [013]-[069]), corresponding to U.S. patent application Ser. No. 15/776,255, the entirety of which is herein incorporated by reference.

In some embodiments of the methods herein, the composition is administered parenterally.

In some embodiments of the methods herein, the composition is administered intravenously.

In some embodiments of the methods herein, the composition is administered intramuscularly.

In some embodiments of the methods herein, the composition is administered intrathecally.

In some embodiments of the methods herein, the composition is administered subcutaneously.

In some embodiments of the methods herein, the composition is administered intraperitoneally.

In some embodiments of the methods herein, the composition is dried prior to the administration step. In some embodiments of the method, the composition is freeze-dried prior to the administration step. In some embodiments of the method, the composition is rehydrated following the drying or freeze-drying step.

In some embodiments, the anticoagulant is selected from the group consisting of an anti-factor IIa agent such as dabigatran (e.g., PRADAXA®), argatroban, or hirudin; an anti-factor Xa agent such as rivaroxaban (e.g., XARELTO®), apixaban (e.g., ELIQUIS®), edoxaban (e.g., SAVAYSA®), or fondaparinux (e.g., ARIXTRA®); a traditional anticoagulant such as warfarin (e.g., COUMADIN®) and heparin/LMWH (low molecular weight heparins); supplements such as herbal supplements, and a combination thereof. Examples of supplements include garlic, coenzyme CoQ10, glucosamine, glucosamine-condroitin sulfate. A non-limiting example of an herbal supplement is garlic.

In some embodiments, the anticoagulant is dabigatran (e.g., PRADAXA®).

In some embodiments, the anticoagulant is argatroban.

In some embodiments, the anticoagulant is hirudin.

In some embodiments, the anticoagulant is rivaroxaban (e.g., XARELTO®).

In some embodiments, the anticoagulant is apixaban (e.g., ELIQUIS®).

In some embodiments, the anticoagulant is edoxaban (e.g., SAVAYSA®).

In some embodiments, the anticoagulant is fondaparinux (e.g., ARIXTRA®).

In some embodiments, the anticoagulant is heparin or a low molecular weight heparin (LMWH).

In some embodiments, the anticoagulant is warfarin (e.g., COUMADIN®).

In some embodiments, the anticoagulant is tifacogin.

In some embodiments, the anticoagulant is Factor VIIai.

In some embodiments, the anticoagulant is SB249417.

In some embodiments, the anticoagulant is pegnivacogin (with or without anivamersen).

In some embodiments, the anticoagulant is TTP889.

In some embodiments, the anticoagulant is idraparinux.

In some embodiments, the anticoagulant is idrabiotaparinux.

In some embodiments, the anticoagulant is SR23781A.

In some embodiments, the anticoagulant is apixaban.

In some embodiments, the anticoagulant is betrixaban.

In some embodiments, the anticoagulant is lepirudin.

In some embodiments, the anticoagulant is bivalirudin.

In some embodiments, the anticoagulant is ximelagatran.

In some embodiments, the anticoagulant is phenprocoumon.

In some embodiments, the anticoagulant is acenocoumarol.

In some embodiments, the anticoagulant an indandione.

In some embodiments, the anticoagulant is fluindione.

In some embodiments, the anticoagulant is a supplement.

In some embodiments, the anticoagulant is an herbal supplement.

In some embodiments, rehydrating the composition comprising platelets such as lyophilized platelets or platelet derivatives comprises adding to the platelets an aqueous liquid. In some embodiments, the aqueous liquid is water. In some embodiments, the aqueous liquid is an aqueous solution (e.g., a buffer). In some embodiments, the aqueous liquid is a saline solution. In some embodiments, the aqueous liquid is a suspension.

In some embodiments, the rehydrated platelets or platelet derivatives (e.g., thrombosomes) have coagulation factor levels showing all individual factors (e.g., Factors VII, VIII and IX) associated with blood clotting at 40 international units (IU) or greater.

In some embodiments, the platelets or platelet derivatives (e.g., thrombosomes) have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets or platelet derivatives (e.g., thrombosomes), have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the platelets such as lyophilized platelets or platelet derivatives (e.g., thrombosomes) have a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets such as lyophilized platelets or platelet derivatives (e.g., thrombosomes), have a particle size in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets such as lyophilized platelets or platelet derivatives (e.g., thrombosomes), are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets such as lyophilized platelets or platelet derivatives (e.g., thrombosomes) are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, platelets are isolated, for example in a liquid medium, prior to treating a subject.

In some embodiments, platelets are donor-derived platelets. In some embodiments, platelets are obtained by a process that comprises an apheresis step. In some embodiments, platelets are pooled platelets.

In some embodiments, platelets are pooled from a plurality of donors. Such platelets pooled from a plurality of donors may be also referred herein to as pooled platelets. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35. Pooled platelets can be used to make any of the compositions described herein.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, platelets are prepared prior to treating a subject as described herein. In some embodiments, the platelets are lyophilized. In some embodiments, the platelets are cryopreserved.

In some embodiments, the platelets or pooled platelets may be acidified to a pH of about 6.0 to about 7.4 prior to the incubation with the incubating agent. In some embodiments, the method comprises acidifying the platelets to a pH of about 6.5 to about 6.9. In some embodiments, the method comprises acidifying the platelets to a pH of about 6.6 to about 6.8. In some embodiments, the acidifying comprises adding to the pooled platelets a solution comprising Acid Citrate Dextrose (ACD).

In some embodiments, the platelets are isolated prior to the incubation with the incubating agent. In some embodiments, the method further comprises isolating platelets by using centrifugation. In some embodiments, the centrifugation occurs at a relative centrifugal force (RCF) of about 1000×g to about 2000×g. In some embodiments, the centrifugation occurs at relative centrifugal force (RCF) of about 1300×g to about 1800×g. In some embodiments, the centrifugation occurs at relative centrifugal force (RCF) of about 1500×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

An incubating agent can include any appropriate components. In some embodiments, the incubating agent may comprise a liquid medium. In some embodiments the incubating agent may comprise one or more salts selected from phosphate salts, sodium salts, potassium salts, calcium salts, magnesium salts, and any other salt that can be found in blood or blood products, or that is known to be useful in drying platelets, or any combination of two or more of these.

In some embodiments, the incubating agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the incubating agent includes from about 0.5 mM to about 100 mM of the one or more salts. In some embodiments, the incubating agent includes from about 0.5 mM to about 100 mM (e.g., about 0.5 to about 2 mM, about 2 mM to about 90 mM, about 2 mM to about 6 mM, about 50 mM to about 100 mM, about 60 mM to about 90 mM, about 70 to about 85 mM) about of the one or more salts. In some embodiments, the incubating agent includes about 5 mM, about 60 mM, about 65 mM, about 70 mM, about 75 mM, or about 80 mM of the one or more salts. In some embodiments, the incubating agent comprises one or more salts selected from calcium salts, magnesium salts, and a combination of the two, in a concentration of about 0.5 mM to about 2 mM.

Preferably, these salts are present in the composition comprising platelets or platelet derivatives, such as freeze-dried platelets, at an amount that is about the same as is found in whole blood.

In some embodiments, the incubating agent further comprises a carrier protein. In some embodiments, the carrier protein comprises human serum albumin, bovine serum albumin, or a combination thereof. In some embodiments, the carrier protein is present in an amount of about 0.05% to about 1.0% (w/v).

The incubating agent may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES). In some embodiments, the incubating agent includes one or more buffers, e.g., N-2-hydroxyethylpiperazine-N′-2-ethanesulfonic acid (HEPES), or sodium-bicarbonate (NaHCO₃). In some embodiments, the incubating agent includes from about 5 to about 100 mM of the one or more buffers. In some embodiments, the incubating agent includes from about 5 to about 50 mM (e.g., from about 5 mM to about 40 mM, from about 8 mM to about 30 mM, about 10 mM to about 25 mM) about of the one or more buffers. In some embodiments, the incubating agent includes about 10 mM, about 20 mM, about 25 mM, or about 30 mM of the one or more buffers.

In some embodiments, the incubating agent includes one or more saccharides, such as monosaccharides and disaccharides, including sucrose, maltose, trehalose, glucose, mannose, dextrose, and xylose. In some embodiments, the saccharide is a monosaccharide. In some embodiments, the saccharide is a disaccharide. In some embodiments, the saccharide is a monosaccharide, a disaccharide, or a combination thereof. In some embodiments, the saccharide is a non-reducing disaccharide. In some embodiments, the saccharide comprises sucrose, maltose, trehalose, glucose (e.g., dextrose), mannose, or xylose. In some embodiments, the saccharide comprises trehalose. In some embodiments, the incubating agent comprises a starch. In some embodiments, the incubating agent includes polysucrose, a polymer of sucrose and epichlorohydrin. In some embodiments, the incubating agent includes from about 10 mM to about 1,000 mM of the one or more saccharides. In some embodiments, the incubating agent includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM. In some embodiments, the one or more saccharides are the lyophilizing agent; for example, in some embodiments, the lyophilizing agent comprises trehalose, polysucrose, or a combination thereof.

In some embodiments the composition comprising platelets or platelet derivatives, (e.g., thrombosomes), may comprise one or more of water or a saline solution. In some embodiments the composition comprising platelets or platelet derivatives, such as freeze-dried platelets, may comprise DMSO.

In some embodiments, the incubating agent comprises an organic solvent, such as an alcohol (e.g., ethanol). In such an incubating agent, the amount of solvent can range from 0.1% to 5.0% (v/v). In some embodiments, the organic solvent can range from about 0.1% (v/v) to about 5.0% (v/v), such as from about 0.3% (v/v) to about 3.0% (v/v), or from about 0.5% (v/v) to about 2% (v/v).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. In some embodiments the organic solvent is selected from the group consisting of ethanol, acetic acid, acetone, acetonitrile, dimethylformamide, dimethyl sulfoxide (DMSO), dioxane, methanol, n-propanol, isopropanol, tetrahydrofuran (THF), N-methyl pyrrolidone, dimethylacetamide (DMAC), or combinations thereof. In some embodiments, the organic solvent comprises ethanol, DMSO, or a combination thereof. The presence of organic solvents, such as ethanol, can be beneficial in the processing of platelets, platelet derivatives, or thrombosomes (e.g., freeze-dried platelet derivatives).

In some embodiments the incubating agent is incubated into the platelets in the presence of an aqueous medium. In some embodiments the incubating agent is incubated in the presence of a medium comprising DMSO.

In some embodiments, one or more other components may be incubated in the platelets. Exemplary components may include Prostaglandin E1 or Prostacyclin and or EDTA/EGTA to prevent platelet aggregation and activation during the incubating process.

Non-limiting examples of incubating agent compositions that may be used are shown in Tables 1-5.

TABLE 1
Buffer

| Component | Concentration (mM unless otherwise specified) |
|---|---|
| NaCl | 75.0 |
| KCl | 4.8 |
| HEPES | 9.5 |
| NaHCO₃ | 12.0 |
| Dextrose | 3 |
| Trehalose | 100 |
| Ethanol (optional) | 1% (v/v) |

TABLE 2
Buffer A

| Component | Concentration (mM unless specified otherwise) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

TABLE 3
Buffer B

| Component | Concentration (mM unless otherwise specified) |
|---|---|
| Buffer and Salts | Table 4 (below) |
| BSA | 0.35% |
| Dextrose | 5 |
| pH | 7.4 |

Table 3. Buffer B can used when incubating platelets, e.g., for flow cytometry. Such an incubation can be done at room temperature in the dark. Albumin is an optional component of Buffer B.

TABLE 4
Concentration of HEPES and of Salts in Buffer B

| Component | Concentration (mM unless otherwise specified) |
|---|---|
| HEPES | 25 |
| NaCl | 119 |
| KCl | 5 |
| CaCl₂ | 2 |
| MgCl₂ | 2 |
| glucose | 6 g/l |

Table 4 is another exemplary incubating agent. The pH can be adjusted to 7.4 with NaOH. Albumin is an optional component of Buffer B.

TABLE 5
Tyrode's HEPES Buffer (plus PGE1)

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostagalandin E1 | 1 μg/ml |
| (PGE1) | |

Table 5 is another exemplary incubating agent.

In some embodiments, platelets (e.g., apheresis platelets, platelets isolated from whole blood, pooled platelets, or a combination thereof) are incubated with the incubating agent for different durations at or at different temperatures from 15-45° C., or about 37° C.

In some embodiments, platelets (e.g., apheresis platelets, platelets isolated from whole blood, pooled platelets, or a combination thereof) form a suspension in an incubating agent comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

The platelets (e.g., apheresis platelets, platelets isolated from whole blood, pooled platelets, or a combination thereof) may be incubated with the incubating agent for different durations, such as, for example, for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the platelets may be incubated with the incubating agent for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the platelets may be incubated with the incubating agent for from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs. In some embodiments, the platelets, the platelet derivatives, or the thrombosomes are incubated with the incubating agent for a period of time of 5 minutes to 48 hours, such as 10 minutes to 24 hours, such as 20 minutes to 12 hours, such as 30 minutes to 6 hours, such as 1 hour minutes to 3 hours, such as about 2 hours.

In some embodiments, the platelets (e.g., apheresis platelets, platelets isolated from whole blood, pooled platelets, or a combination thereof) are incubated with the incubating agents at different temperatures. In embodiments, incubation is conducted at 37° C. In certain embodiments, incubation is performed at 4° C. to 45° C., such as 15° C. to 42° C. For example, in embodiments, incubation is performed at 35° C. to 40° C. (e.g., 37° C.) for 110 to 130 (e.g., 120) minutes and for as long as 24-48 hours. In some embodiments, the platelets are incubated with the incubating agent for different durations as disclosed herein, and at temperatures from 15-45° C., or about 37° C.

In some embodiments, platelets (e.g., apheresis platelets, platelets isolated from whole blood, pooled platelets, or a combination thereof) are loaded with one or more active agents. In some embodiments, the platelets can be loaded with an anti-fibrinolytic agent. Non-limiting examples of anti-fibrinolytic agents include F-aminocaproic acid (EACA), tranexamic acid, aprotinin, aminomethylbenzoic acid, and fibrinogen.

Loading platelets (e.g., apheresis platelets, platelets isolated from whole blood, pooled platelets, or a combination thereof) with an active agent (e.g., an anti-fibrinolytic agent) can be performed by any appropriate method. See, for example, PCT Publication Nos. WO2020113090A1, WO2020113101A1, WO2020113035A1, and WO2020112963A1. Generally, the loading includes contacting the platelets with the anti-fibrinolytic agent. In some embodiments, the loading can be performed by combining the active agent with the incubating agent. In some embodiments, the loading can be performed in a separate step from the incubating step. For example, the loading can be performed in a step prior to the incubation step. In some such embodiments, the active agent can be supplied to the platelets as a solution or suspension in any of the incubation agents described herein, which may or may not be the same as the incubating agent used in the incubating step. In some embodiments, the loading step can be performed during the incubation step. In some such embodiments, the active agent can be added to the incubation agent (e.g., as a solid or in a solution or suspension) during the incubation). In some embodiments, the loading step can be performed in a step following the incubation step. In some such embodiments, be supplied to the platelets as a solution or suspension in any of the incubation agents described herein, which may or may not be the same as the incubating agent used in the incubating step.

An active agent can be applied to the platelets in any appropriate concentration. In some embodiments, an active agent can be applied to the platelets (e.g., as part of the incubating agent or another solution or suspension) in a concentration of about 1 μM to about 100 mM (e.g., about 1 μM to about 10 μm, about 1 μM to about 50 μM, about 1 μM to about 100 μM, about 1 μM to about 500 μM, about 1 μM to about 1 mM, about 1 μM to about 10 mM, about 1 μM to about 25 mM, about 1 μM to about 50 mM, about 1 μM to about 75 mM, about 10 μM to about 100 mM, about 50 μM to about 100 mM, about 100 μM to about 100 mM, about 500 μM to about 100 mM, about 1 mM to about 100 mM, about 10 mM to about 100 mM, about 25 mM to about 100 mM, about 50 mM to about 100 mM, about 75 mM to about 100 mM, about 10 μM to about 100 mM, about 200 μM to about 1 mM, about 800 μM to about 900 μM, about 400 μM to about 800 μM, about 500 μM to about 700 μM, about 600 μM, about 5 mM to about 85 mM, about 20 mM to about 90 mM, about 25 mM to about 75 mM, about 30 mM to about 90 mM, about 35 mM to about 65 mM, about 40 mM to about 60 mM, about 50 mM to about 60 mM, about 40 mM to about 70 mM, about 45 mM to about 55 mM, or about 50 mM).

In some embodiments, the method further comprises drying the platelets. In some embodiments, the drying step comprises lyophilizing the platelets. In some embodiments, the drying step comprises freeze-drying the platelets. In some embodiments, the method further comprises rehydrating the platelets obtained from the drying step.

In some embodiments, the platelets are cold stored, cryopreserved, or lyophilized (e.g., to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and 8,097,403. An exemplary spray-drying method includes: combining nitrogen, as a drying gas, with a incubating agent according to the present disclosure, then introducing the mixture into GEA Mobile Minor spray dryer from GEA Processing Engineering, Inc. (Columbia Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | Step | Temp. Set | | Type | Duration | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the platelets that are obtained as disclosed herein, such as the step of freeze-drying the platelets that are obtained as disclosed herein, comprises incubating the platelets with a lyophilizing agent (e.g., a non-reducing disaccharide). Accordingly, in some embodiments, the methods for preparing platelets further comprise incubating the platelets with a lyophilizing agent. In some embodiments the lyophilizing agent is a saccharide. In some embodiments the saccharide is a disaccharide, such as a non-reducing disaccharide.

In some embodiments, the platelets are incubated with a lyophilizing agent for a sufficient amount of time and at a suitable temperature to incubate the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrolidone (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., polysucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

Within the process provided herein for making the compositions provided herein, addition of the lyophilizing agent can be the last step prior to drying. However, in some embodiments, the lyophilizing agent is added at the same time or before other components of the composition, such as a salt, a buffer, optionally a cryoprotectant, or other components. In some embodiments, the lyophilizing agent is added to the incubating agent, thoroughly mixed to form a drying solution, dispensed into a drying vessel (e.g., a glass or plastic serum vial, a lyophilization bag), and subjected to conditions that allow for drying of the solution to form a dried composition.

The step of incubating the platelets with a cryoprotectant can include incubating the platelets for a time suitable for loading, as long as the time, taken in conjunction with the temperature, is sufficient for the cryoprotectant to come into contact with the platelets and, preferably, be incorporated, at least to some extent, into the platelets. In embodiments, incubation is carried out for about 1 minute to about 180 minutes or longer.

The step of incubating the platelets with a cryoprotectant can include incubating the platelets and the cryoprotectant at a temperature that, when selected in conjunction with the amount of time allotted, is suitable for incubating. In general, the composition is incubated at a temperature above freezing for at least a sufficient time for the cryoprotectant to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, dried platelets or platelet derivatives (e.g., thrombosomes) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Thus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets or platelet derivatives (e.g., thrombosomes) for at least 30 minutes at a temperature higher than 30° C.; heating the dried platelets or platelet derivatives (e.g., thrombosomes) for at least 10 hours at a temperature higher than 50° C.; heating the dried platelets or platelet derivatives (e.g., thrombosomes) for at least 18 hours at a temperature higher than 75° C.; and heating the dried platelets or platelet derivatives (e.g., thrombosomes) for 24 hours at 80° C. In some embodiments, heating can be performed in sealed container, such as a capped vial. In some embodiments, a sealed container be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets or platelet derivatives (e.g., thrombosomes).

In some embodiments, the platelets or platelet derivatives (e.g., thrombosomes) prepared as disclosed herein by a process comprising incubation with an incubating agent have a storage stability that is at least about equal to that of the platelets prior to the incubation.

In some embodiments, the method further comprises cryopreserving the platelets or platelet derivatives prior to administering the platelets or platelet derivatives (e.g., with an incubating agent, e.g., an incubating agent described herein).

In some embodiments, the method further comprises drying a composition comprising platelets or platelet derivatives, (e.g., with an incubating agent e.g., an incubating agent described herein) prior to administering the platelets or platelet derivatives (e.g., thrombosomes). In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with an incubating agent e.g., an incubating agent described herein) prior to administering the platelets or platelet derivatives (e.g., thrombosomes) In some embodiments, the method may further comprise heating the composition following the freeze-drying step. In some embodiments, the method may further comprise rehydrating the composition following the freeze-drying step or the heating step.

In some embodiments, the method further comprises cold storing the platelets, platelet derivatives, or the thrombosomes prior to administering the platelets, platelet derivatives, or thrombosomes (e.g., with an incubating agent, e.g., an incubating agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets or platelet derivatives (e.g., thrombosomes) (e.g., with an incubating agent e.g., an incubating agent described herein) prior to administering the platelets or platelet derivatives (e.g., thrombosomes). For example, in some embodiments, the method further comprises drying (e.g., freeze-drying) a composition comprising platelets or platelet derivatives (e.g., with an incubating agent e.g., an incubating agent described herein) (e.g., to form thrombosomes) prior to administering the platelets or platelet derivatives (e.g., thrombosomes). In some embodiments, the method may further comprise rehydrating the composition obtained from the drying step.

In some embodiments, provided herein is composition comprising platelets such as lyophilized platelets or platelet derivatives (e.g., thrombosomes), polysucrose and trehalose made by the process of obtaining fresh platelets, optionally incubating the platelets in DMSO, isolating the platelets by centrifugation, resuspending the platelets in an incubating agent which comprises trehalose and ethanol thereby forming a first mixture, incubating the first mixture, mixing polysucrose with the first mixture, thereby forming a second mixture, and lyophilizing the second mixture to form a freeze dried composition comprising platelets or platelet derivatives (e.g., thrombosomes), polysucrose and trehalose.

In some embodiments, provided herein is a method of making a freeze-dried platelet composition comprising platelets or platelet derivatives (e.g., thrombosomes), polysucrose and trehalose comprising obtaining fresh platelets, optionally incubating the platelets in DMSO, isolating the platelets by centrifugation, resuspending the platelets in a incubating agent which comprises trehalose and ethanol thereby forming a first mixture, incubating the first mixture, mixing polysucrose with the first mixture, thereby forming a second mixture, and lyophilizing the second mixture to form a freeze-dried composition comprising platelets or platelet derivatives (e.g., thrombosomes), polysucrose and trehalose.

In some embodiments, provided herein is a process for making freeze-dried platelets, the process comprising incubating isolated platelets in the presence of at least one saccharide under the following conditions: a temperature of from 20° C. to 42° C. for about 10 minutes to about 180 minutes, adding to the platelets at least one cryoprotectant, and lyophilizing the platelets, wherein the process optionally does not include isolating the platelets between the incubating and adding steps, and optionally wherein the process does not include exposing the platelets to a platelet activation inhibitor. The cryoprotectant can be a polysugar (e.g., polysucrose). The process can further include heating the lyophilized platelets at a temperature of 70° C. to 80° C. for 8 to 24 hours. The step of adding to the platelets at least one cryoprotectant can further include exposing the platelets to ethanol. The step of incubating isolated platelets in the presence of at least one saccharide can include incubating in the presence of at least one saccharide. The step of incubating isolated platelets in the presence of at least one saccharide can include incubating in the presence of at least one saccharide. The conditions for incubating can include incubating for about 100 minutes to about 150 minutes. The conditions for incubating can include incubating for about 110 minutes to about 130 minutes. The conditions for incubating can include incubating for about 120 minutes. The conditions for incubating can include incubating at 35° C. to 40° C. The conditions for incubating can include incubating at 37° C. The conditions for incubating can include incubating at 35° C. to 40° C. for 110 minutes to 130 minutes. The conditions for incubating can include incubating at 37° C. for 120 minutes. The at least one saccharide can be trehalose, sucrose, or both trehalose and sucrose. The at least one saccharide can be trehalose. The at least one saccharide can be sucrose.

In some embodiments, provided herein is a method of preparing freeze-dried platelets, the method including providing platelets, suspending the platelets in a salt buffer that includes about 100 mM trehalose and about 1% (v/v) ethanol to make a first composition, incubating the first composition at about 37° C. for about 2 hours, adding polysucrose (e.g., polysucrose 400) to a final concentration of about 6% (w/v) to make a second composition, lyophilizing the second composition to make freeze-dried platelets, and heating the freeze-dried platelets at 80° C. for 24 hours.

Specific embodiments disclosed herein may be further limited in the claims using “consisting of” or “consisting essentially of” language.

### EXEMPLARY EMBODIMENTS

Embodiment 1 is a method of treating a coagulopathy in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 2 is a method of treating a coagulopathy in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 3 is a method of restoring normal hemostasis in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 4 is a method of restoring normal hemostasis in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 5 is a method of preparing a subject for surgery, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 6 is a method of preparing a subject for surgery, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 7 is the method of any one of embodiments 5-6, wherein the surgery is an emergency surgery.

Embodiment 8 is the method of any one of embodiments 5-6, wherein the surgery is a scheduled surgery.

Embodiment 9 is the method of any one of embodiments 1-8, wherein the subject has been treated or is being treated with an anticoagulant.

Embodiment 10 is the method of embodiment 9, wherein treatment with the anticoagulant is stopped.

Embodiment 11 is the method of embodiment 9, wherein treatment with the anticoagulant is continued.

Embodiment 12 is a method of ameliorating the effects of an anticoagulant in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 13 is a method of ameliorating the effects of an anticoagulant in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

Embodiment 14 is the method of embodiment 12 or embodiment 13, wherein the effects of the anticoagulant are the result of an overdose of the anticoagulant.

Embodiment 15 is the method of any one of embodiments 1-14, wherein the composition further comprises an anti-fibrinolytic agent.

Embodiment 16 is the method of embodiment 15, wherein the anti-fibrinolytic agent is selected from the group consisting of F-aminocaproic acid (EACA), tranexamic acid, aprotinin, aminomethylbenzoic acid, fibrinogen, and a combination thereof.

Embodiment 17 is the method of embodiment 15 or embodiment 16, wherein the platelets or platelet derivatives are loaded with the anti-fibrinolytic agent.

Embodiment 18 is the method of any one of embodiments 9-17, wherein the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof.

Embodiment 19 is the method of any one of embodiments 9-17, wherein the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof.

Embodiment 20 is the method of embodiment 18 or embodiment 19, wherein the anticoagulant is warfarin.

Embodiment 21 is the method of embodiment 18 or embodiment 19, wherein the anticoagulant is heparin.

Embodiment 22 is the method of any one of embodiments 1-21, wherein before the administering, the subject had an INR of at least 4.0.

Embodiment 23 is the method of embodiment 22, wherein after the administering, the subject has an INR of 3.0 or less.

Embodiment 24 is the method of embodiment 22, wherein after the administering, the subject has an INR of 2.0 or less.

Embodiment 25 is the method of any one of embodiments 1-21, wherein before the administering, the subject had an INR of at least 3.0.

Embodiment 26 is the method of embodiment 25, wherein after the administering, the subject has an INR of 2.0 or less.

Embodiment 27 is the method of any one of embodiments 1-26, wherein administering comprises administering topically.

Embodiment 28 is the method of any one of embodiments 1-26, wherein administering comprises administering parenterally.

Embodiment 29 is the method of any one of embodiments 1-26, wherein administering comprises administering intravenously.

Embodiment 30 is the method of any one of embodiments 1-26, wherein administering comprises administering intramuscularly.

Embodiment 31 is the method of any one of embodiments 1-26, wherein administering comprises administering intrathecally.

Embodiment 32 is the method of any one of embodiments 1-26, wherein administering comprises administering subcutaneously.

Embodiment 33 is the method of any one of embodiments 1-26, wherein administering comprises administering intraperitoneally.

Embodiment 34 is the method of any one of embodiments 1-33, wherein the composition is dried prior to the administration step.

Embodiment 35 is the method of embodiment 34, wherein the composition is rehydrated following the drying step.

Embodiment 36 is the method of any one of embodiments 1-34, wherein the composition is freeze-dried prior to the administration step.

Embodiment 37 is the method of embodiment 36, wherein the composition is rehydrated following the freeze-drying step.

Embodiment 38 is the method of any one of embodiments 1-37, wherein the incubating agent comprises one or more salts selected from phosphate salts, sodium salts, potassium salts, calcium salts, magnesium salts, and a combination of two or more thereof.

Embodiment 39 is the method of any one of embodiments 1-38, wherein the incubating agent comprises a carrier protein.

Embodiment 40 is the method of any one of embodiments 1-39, wherein the buffer comprises HEPES, sodium bicarbonate (NaHCO₃), or a combination thereof.

Embodiment 41 is the method of any one of embodiments 1-40, wherein the composition comprises one or more saccharides.

Embodiment 42 is the method of embodiment 41, wherein the one or more saccharides comprise trehalose.

Embodiment 43 is the method of embodiment 41 or embodiment 42, wherein the one or more saccharides comprise polysucrose.

Embodiment 44 is the method of any one of embodiments 41-43, wherein the one or more saccharides comprise dextrose.

Embodiment 45 is the method of any one of embodiments 1-44, wherein the composition comprises an organic solvent.

Embodiment 46 is the method of any one of embodiments 1-45, wherein the platelets or platelet derivatives comprise thrombosomes.

### Examples

### Example 1

The results that follow demonstrate the impact of the thrombosomes product in an in vitro model for patients taking warfarin, a common anticoagulant drug. Warfarin inhibits the synthesis of numerous hemostatic plasma proteins in the liver that are dependent on vitamin K.

Thrombosomes and other lyophilized platelet products are designed for infusion into a patient's bloodstream following diagnosis of trauma or hemostatic failure. In the following Examples modeling patients using warfarin, thrombosomes were introduced first into a plasma-based system, followed by a whole-blood system in Example 2 to more closely mimic conditions in vivo.

In the plasma model, thrombosomes demonstrated a noticeable improvement in thrombin generation (TGA) and thromboelastography (TEG) assays.

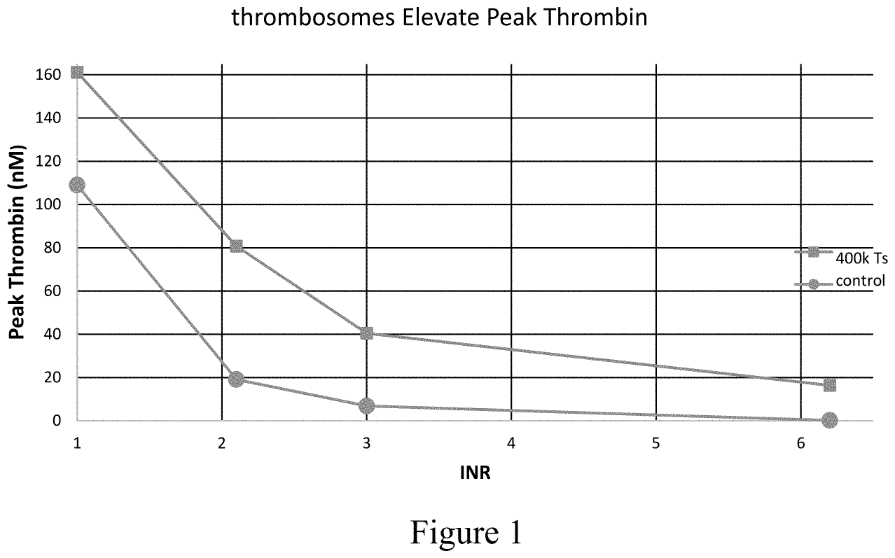

The samples used in the plasma model were prepared by combining 1:1 volumes of warfarin plasma (source: George King Biomedical, at various INR values) or platelet-rich plasma (PRP) and Control Buffer detailed below in Table 6, with or without rehydrated thrombosomes at the concentrations indicated in FIGS. 1-3. Warfarin plasma was obtained from the blood drawn from patients using the drug. Because warfarin inhibits the biological synthesis of hemostatic proteins, it cannot be added ex vivo. Thrombosomes were prepared consistent with the procedures described in U.S. Pat. No. 8,486,617 (such as, e.g., Examples 1-5) and U.S. Pat. No. 8,097,403 (such as, e.g., Examples 1-3), incorporated herein by reference in their entirety and rehydrated by addition of sterile water.

TABLE 6
Composition of Control Buffer

| Component | Concentration (mg/mL, except where otherwise indicated) | |
|---|---|---|
| NaCl | 6.08 | |
| KCl | 0.28 | |
| HEPES | 2.47 | |
| NaHCO₃ | 0.77 | |
| Dextrose | 0.41 | |
| Trehalose | 28.83 | |
| Ethanol | 0.76% | (v/v) |
| Polysucrose | 6% | (m/v) |

As INR increases, thrombin generation decreases. Across all doses, thrombosomes demonstrate notable improvement in peak thrombin. As thrombosomes show an uptick at each dose level, it is clear that their efficacy is not related to warfarin.

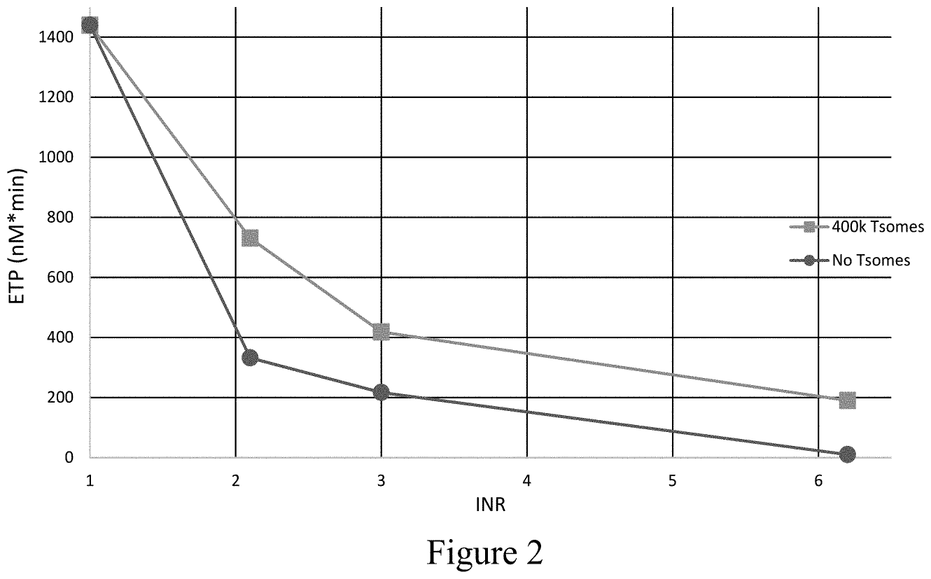

As demonstrated in FIGS. 1 and 2, thrombosomes have a positive impact on thrombin generation (a measure of clotting capability) in a model of warfarin in plasma, assessed in a thrombin generation assay (TGA) as described in Example 3.

Platelet rich plasma sample Preparation

(1) Obtain type O donor whole blood in NaCitrate (blue-top) vacutainer tubes.

(2) Centrifuge the blood at 180×g for 20 minutes.

(3) Carefully pipette off the platelet rich plasma, leaving the buffy coat intact

(4) Take a platelet count in the plasma sample

(5) Supplement appropriate number of platelets per sample to the plasma of choice

In particular, as shown in FIG. 1, peak thrombin generation is improved by adding 400×10³/μL thrombosomes to warfarin plasma. A normal range for peak thrombin was determined to be 66-166 nM, indicating that the uptick in peak thrombin at a normal blood state (INR=1) is not outside reasonable thrombin level. Similarly, FIG. 2 shows that the endogenous thrombin potential (ETP; determined as the area under the curve in the thrombin generation assay) is improved by adding 100×10³/μL thrombosomes to warfarin plasma.

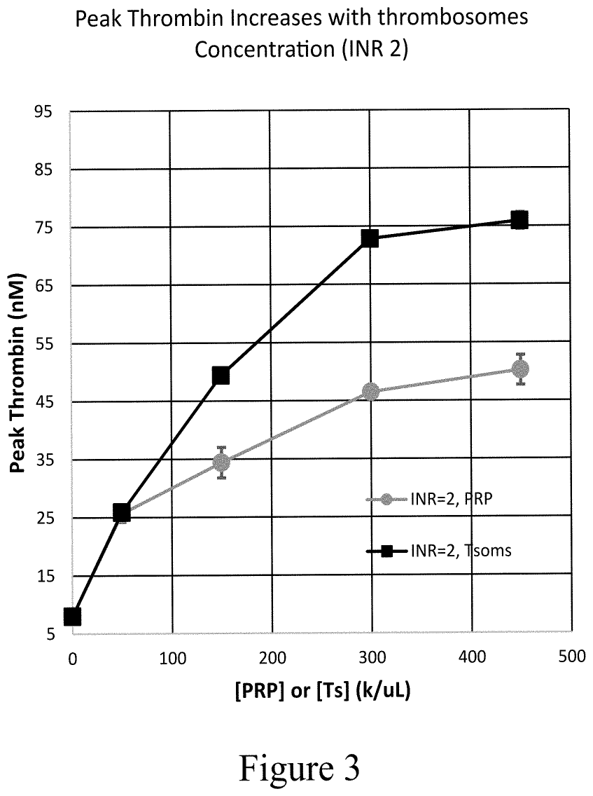

FIG. 3 shows peak thrombin generation by thrombosomes and by platelet-rich plasma (PRP) in INR 2 warfarin plasma. Thrombosomes even generate more thrombin than the platelets, and without being bound by any particular theory or mechanism, this could possibly be due to elevated activation of the thrombosomes. This forecasts a reduction in bleeding in vivo because additional thrombin generation stimulates endogenous clotting mechanisms.

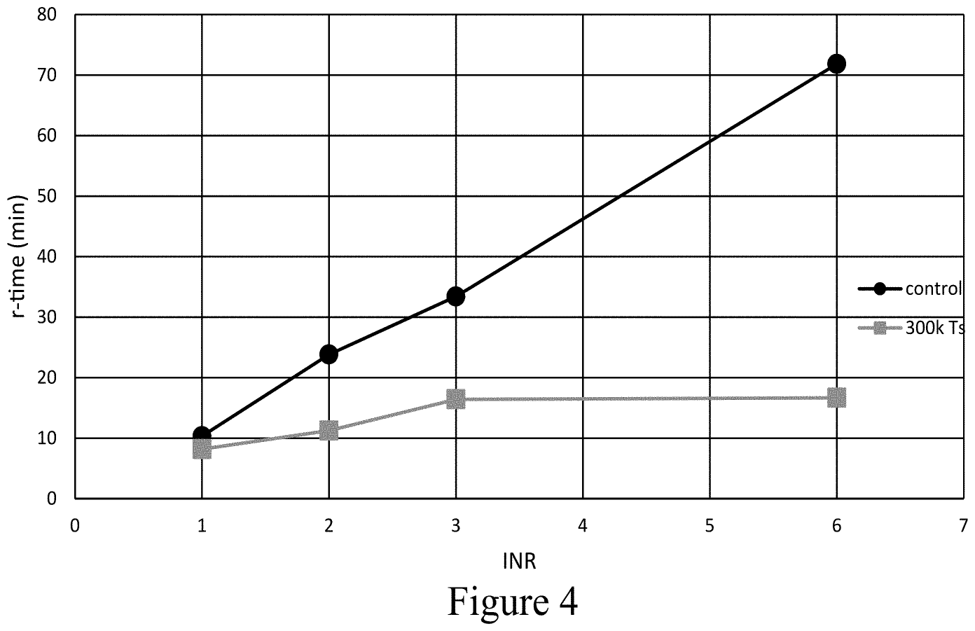

FIG. 4 features data from a thromboelastography (TEG) assay as described in Example 3, a system that measures the viscoelastic properties of blood and plasma. The R-time plotted in FIG. 4 correlates to the speed of clot generation in the plasma model. A reduction in R-time across all warfarin doses was observed with the addition of thrombosomes. In particular, the addition of 300×10³/μL thrombosomes substantially reduced R-time of the warfarin plasma samples (TEG assay). Compared to normal R-time (about 5-10 minutes), the addition of thrombosomes almost completely corrected R-time across all INR levels.

### Example 2: Whole Blood Assays

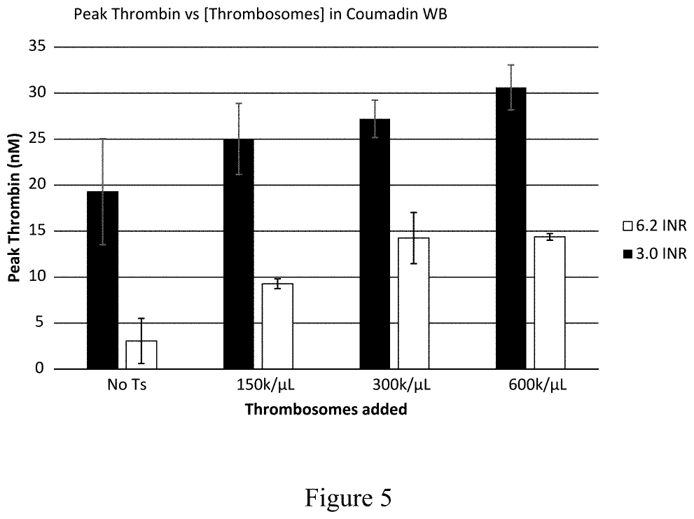

Once the impact in plasma was established, thrombosomes were introduced into a similar warfarin model using donor whole blood. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. To generate comparable anticoagulant conditions, the native plasma of type O donor blood was removed and replaced with warfarin plasma as described in Example 3. TGA assays were performed as described in Example 3. FIG. 5 shows that thrombosomes provide a dose-dependent effect on peak thrombin generation. In FIG. 5, data were collected in the background of whole blood with an endogenous platelet count of 150×10³/μL. An increase in peak thrombin was observed in particular at INR 3.0 and 6.2 in FIG. 5. The roughly 50% increase in peak thrombin (at INR 3) in vitro may translate to significantly lower bleeding in vivo as thrombin generation ultimately determines clot stability.

### Example 3: Procedures

Whole Blood Sample Preparation
(1) Obtain type O donor whole blood in NaCitrate (blue-top) vacutainer tubes.
(2) Centrifuge the blood at 2000×g for 10 minutes.
(3) Carefully pipette off the plasma, leaving the buffy coat intact
(4) Add a volume of HEPES-buffered saline (HBS) equivalent to the removed plasma and gently resuspend the whole blood.
(5) Spin the blood again for 10 minutes at 2000×g.
(6) Carefully remove the supernatant, leaving the buffy coat intact.
(7) Incrementally resuspend the blood in warfarin plasma, normal plasma (function control), or autologous plasma (process control) until the measured hematocrit is equivalent to the hematocrit of the donor's fresh whole blood.
   a. Store at room temperature for up to 4 hours.
(8) Combine 1:1 volume with Control Buffer with or without thrombosomes immediately before running any samples.

Thromboelastography Assay (TEG® 5000 THROMBOELASTOGRAPH® Hemostasis Analyzer System)
(1) Open TEG 5000 assay software and set up instrument according to manufacturer guidelines.
(2) Thaw warfarin plasma in 37° C. water bath for 5 minutes.
(3) Rehydrate thrombosomes with cell culture grade water for 10 minutes then dilute with Control Buffer to 600×10³/μL.
(4) Add 20 μL 0.2M CaCl₂) to empty sample cups.
(5) For each sample, combine 1:1 volumes of Control Buffer with or without thrombosomes and plasma.
(6) Add 340 μL of sample to a cup then quickly load the cup into the device and start the run.
(7) The run is complete when R-time is determined or the run times out.

Thrombin Generation Assay (on Fluoroskan ASCENT®)
(1) Open CAT software; set up instrument; and prepare PRP reagent (including Tissue Factor and some phospholipids), calibrator, and fluoro-buffer according to manufacturer guidelines.
(2) Thaw warfarin or control plasma in 37° C. water bath for 5 minutes.
(3) Rehydrate thrombosomes with cell culture grade water for 10 minutes then dilute with Control Buffer to double target concentration.
(4) For each sample, combine 1:1 volumes of Control Buffer with or without thrombosomes and plasma.
(5) Using a multichannel pipette, add 20 μL of PRP reagent to each well.
(6) Add 80 μL of sample per well. Include one calibrator well for each sample.
(7) Insert plate into tray and inject fluoro-buffer (including a fluorescent-labeled peptide, that when cleaved by thrombin, generates a fluorescent signal) into active wells.
(8) Read plate for 180 minutes at 20 s intervals to capture full thrombin generation profile.

T-TAS®

The T-TAS® instrument was prepared for use according to the manufacturer's instructions. AR Chips (Diapharma Cat. #TC0101) and AR Chip Calcium Corn Trypsin Inhibitor (CaCTI; Diapharma Cat. #TR0101) were warmed to room temperature. 300 uL of rehydrated thrombosomes were transferred to a 1.7 mL microcentrifuge tube and centrifuged at 3900 g×10 minutes to pellet. The thrombosomes pellet was resuspended in George King (GK) pooled normal human plasma or autologous plasma with or without autologous platelets to a concentration of approximately 100,000-450,000/uL, as determined by AcT counts (Beckman Coulter AcT Diff 2 Cell Counter). 20 uL of CaCTI with 480 uL of thrombosomes sample in GK plasma were mixed with gentle pipetting. The sample was loaded and run on the T-TAS® according to the manufacturer's instructions.

Partial Thromboplastin Time (aPTT)

A protocol for measuring aPTT follows.

Turn on instrument; and prepare Reagent 1, Reagent 2, Coag control N and Coag control P according to manufacturer guidelines.

Thaw George King Pooled normal Plasma in 37° C. water bath for 5 minutes.

Place cuvette-strips in the incubation area for prewarming at 37° C. for at least 3 minutes. Dispense a ball to each cuvette.

For each sample, incubate GKP with or without a series of concentrations of Heparin and/or Protamine sulfate for 5 minutes in room temperature.

Dispense 50 μL samples and 50 μL Reagent 1 to each cuvette. Start the timer corresponding to the incubation column for an incubation of 180 seconds.

When the instrument starts to beep, transfer the cuvettes to the test-column area.

Prime the Finnpipette once with 0.025 M CaCl₂.

Activate the Finnpipette by pressing the pipette key. Dispense 50 μL 0.025 M CaCl₂) to each cuvette using Finnpipette.

### Example 4: Comparison to Fresh Platelets

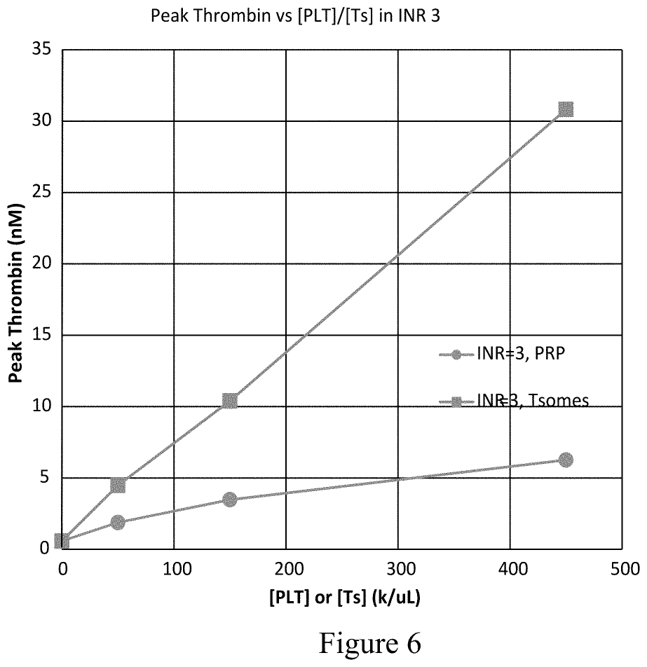

Thrombosomes elicit a specific dose-dependent recovery of thrombin generation in coumadin plasma in a manner superior to fresh platelets. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3. At a dose of INR 3, thrombosomes demonstrate a dose-dependent recovery of peak thrombin (FIG. 6). Additionally, adding Thrombosomes is more effective than an equivalent dose of fresh platelets.

### Example 5: Combination with Fresh Platelets

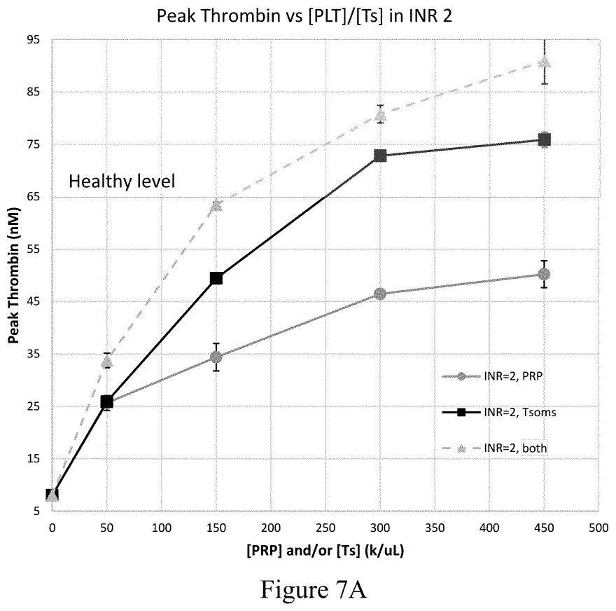

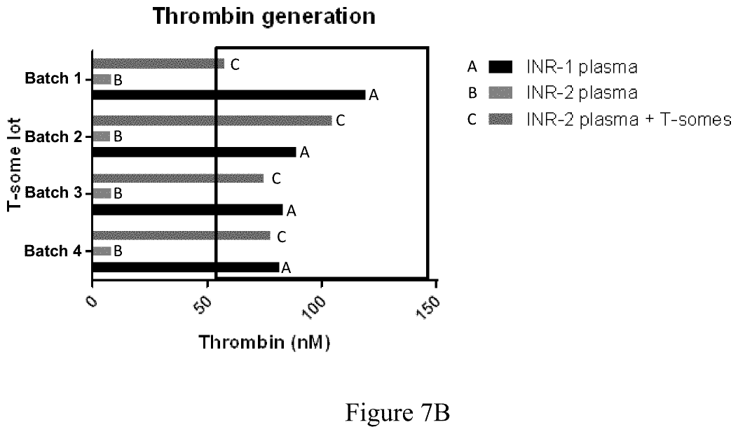

Thrombosomes cooperate with platelets increasing thrombin generation in warfarin plasma. Thrombosomes were prepared consistent with the procedures described in U.S. Pat. No. 8,486,617 (such as, e.g., Examples 1-5) and U.S. Pat. No. 8,097,403 (such as, e.g., Examples 1-3) and rehydrated by addition of sterile water. TGA assays were performed as described in Example 3. Thrombosomes not only show greater efficacy, but also an additive effect with endogenous platelets (FIG. 7A). Note that thrombosomes can push the model patient back into a healthy peak thrombin range (e.g., between about 66 and 166 nM). Note that the ‘both’ line includes the two components in equal amounts in the amounts shown (e.g., at the ‘50’ value on the x-axis, the y-value represents the peak thrombin of a mixture of 50 k platelets from PRP/μL and 50 k thrombosomes/μL.

In addition, different batches of thrombosomes can also push a model patient (INR=2, treated with warfarin) back into a healthy peak thrombin range (FIG. 7B).

### Example 6: Collagen Adhesion

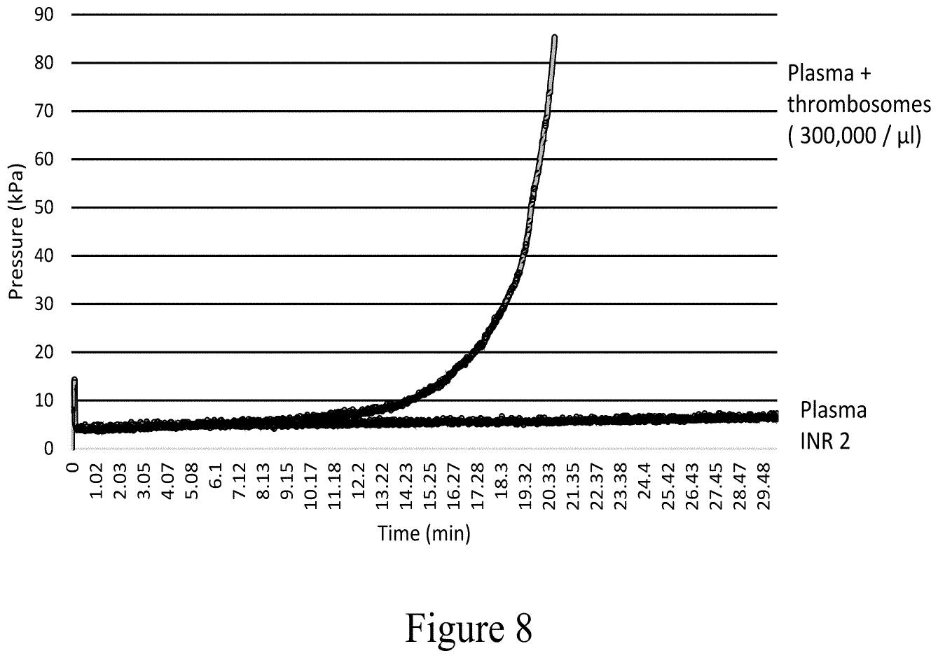

Thrombosomes adhere to and generate fibrin in warfarin plasma using shear-dependent collagen adhesion assay under flow (T-TAS®) (FIG. 8). Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. T-TAS® assays were performed according to Example 3.

### Example 7. Rivaroxaban Results

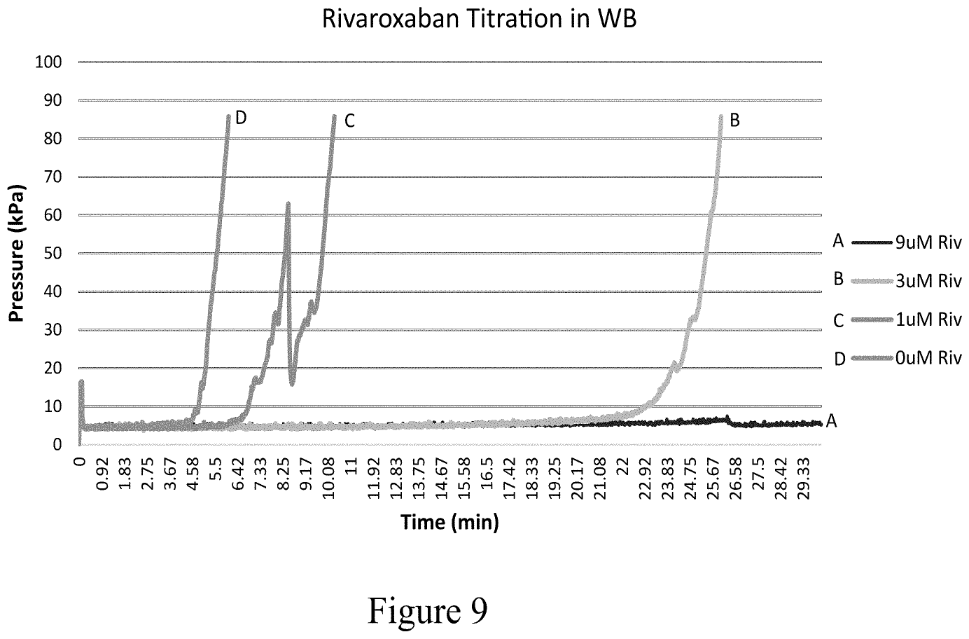

Rivaroxaban (sometimes herein called Riv) dose-response in whole blood was measured using T-TAS®. An AR chip (Collagen+TF) was used. T-TAS® assays were performed according to Example 3. The donor platelets were used at 307 k/μL. A 9 μM dose (a pharmacological dose) inhibits occlusion but not all thrombus formation (FIG. 9, Table 7).

TABLE 7

| [Riv] | | | Occ Spd | AUC |
|---|---|---|---|---|
| (uM) | Occ Time | Occ Start | (kPa/min) | (kPa*min) |
| 0 | 6:04 | 4:56 | 61.8 | 1970 |
| 1 | 10:21 | 7:00 | 20.9 | 1686 |
| 3 | 26:01 | 23:32 | 28.2 | 423 |
| 9 | n/a | n/a | n/a | 13.6 |

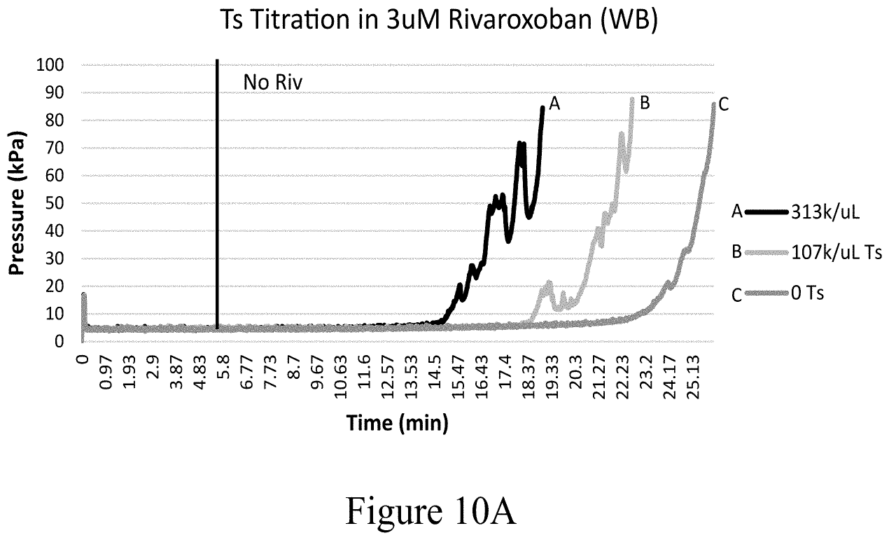

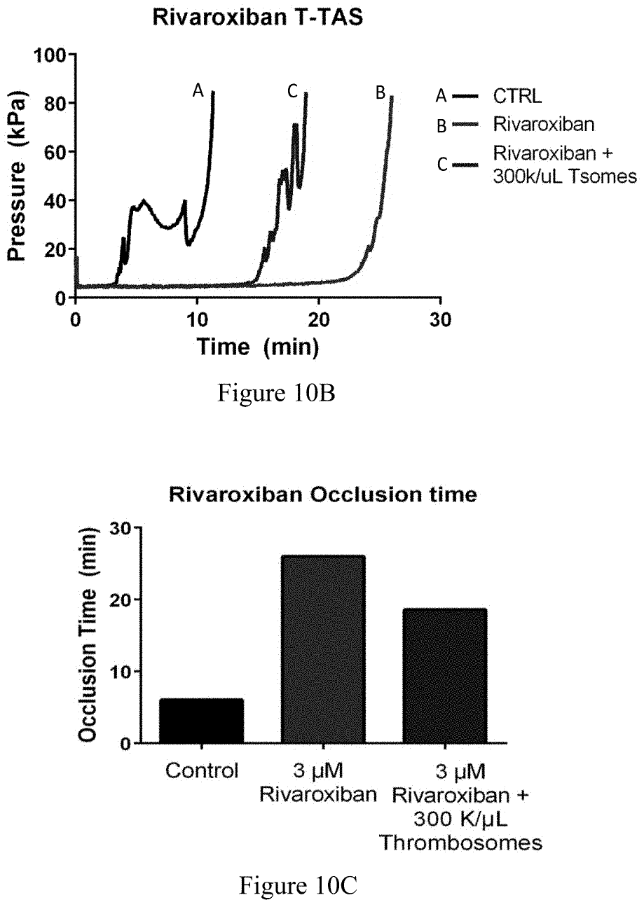

Thrombosomes partially restore thrombus formation in rivaroxaban-anticoagulated whole blood. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. T-TAS® assays were run according to Example 3 using 3 μM rivaroxaban and different concentrations of thrombosomes (FIG. 10A, Table 8). The ‘No Riv’ vertical line indicates the approximate occlusion time of a sample with no added rivaroxaban.

TABLE 8

| [Ts] | | | Occ Spd | AUC |
|---|---|---|---|---|
| (k/uL) | Occ Time | Occ Start | (kPa/min) | (kPa*min) |
| 0 | 26:01 | 23:32 | 28.2 | 423 |
| 108 | 22:39 | 18:49 | 18.3 | 709 |
| 313 | 18:58 | 15:19 | 19.2 | 1033 |

In a similar experiment, T-TAS® assays were run according to Example 3 with no rivaroxaban, 3 μM rivoroxaban, and 3 μM rivoroxaban and 300×103/μL thrombosomes. The pressure over time is shown in FIG. 10B, and the occlusion time is shown in FIG. 10C. Platelet rich plasma that was treated with 3 μM rivaroxaban extended occlusion times from 6.04 to 26.01 minutes on the T-TAS® flow system (collagen and tissue factor coated channel). The addition of 300 k/μL decreased the time back to 18.01 minutes.

### Example 8. Thromboelastography of Warfarin Plasma

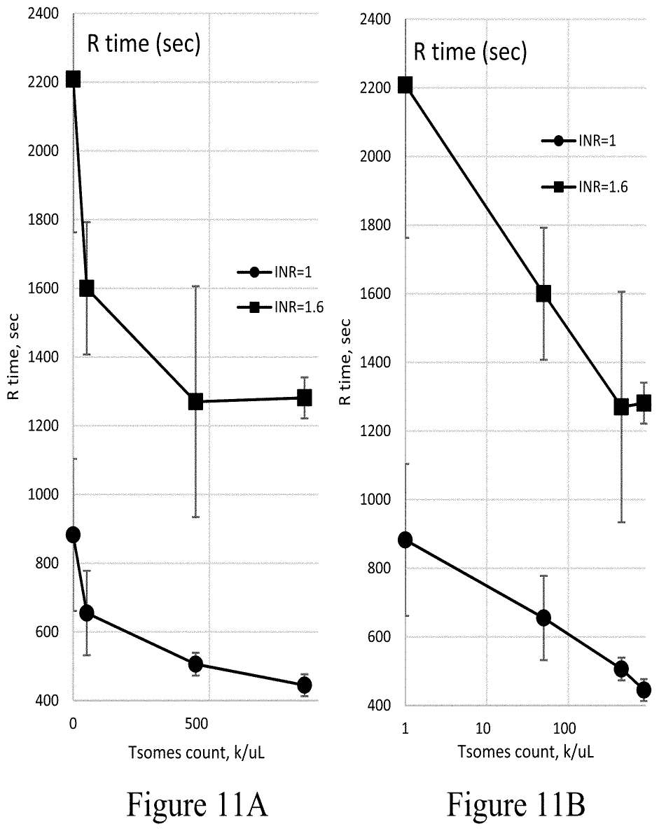

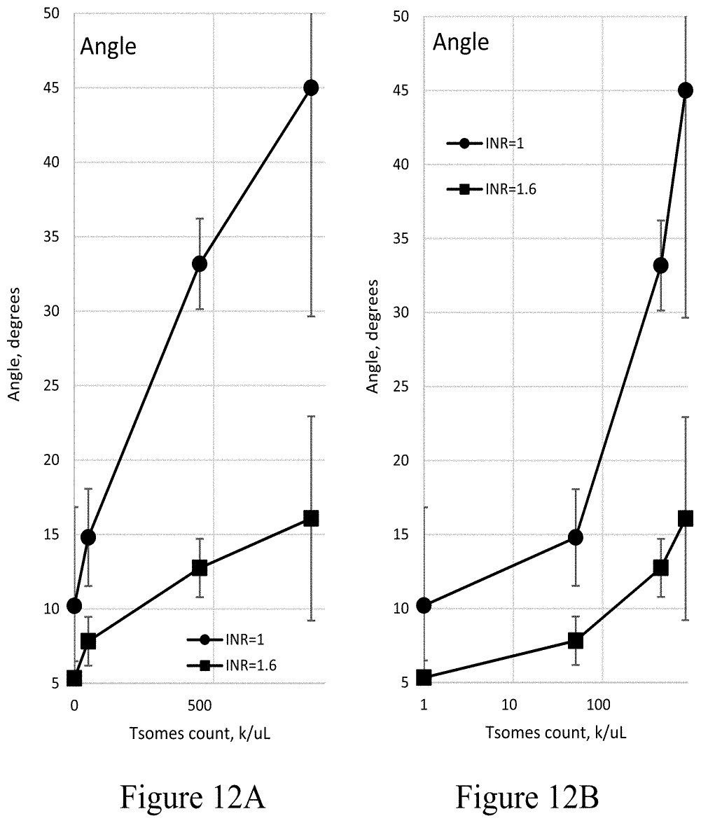

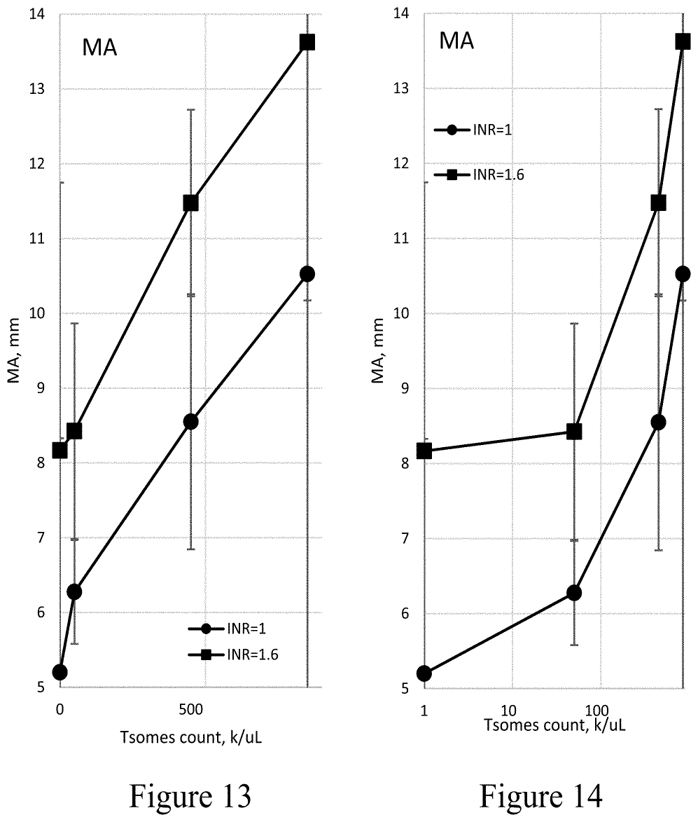

As shown in FIGS. 11-13, the effect of thrombosomes in warfarin plasma (INR=1.6) was tested and compared to standard plasma (INR=1.0), at thrombosome concentrations of 850, 450, 50 and 0 k/uL. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water.

In this experiment, +170 μL of plasma was placed in each cup; +170 μL of thrombosomes or control in each cup; and +20 μL of CaCl₂) (TEG Reagent) in each well.

Each run was performed using single replicate for each condition. Four runs were made in total. Thrombosome dilutions were prepared shortly before each run, and counts were checked immediately after each run was started. The results are shown in FIGS. 11A, 11, 12A, 12B, 13, and 14 (thrombosomes batch 4).

### Example 9. Lag Time

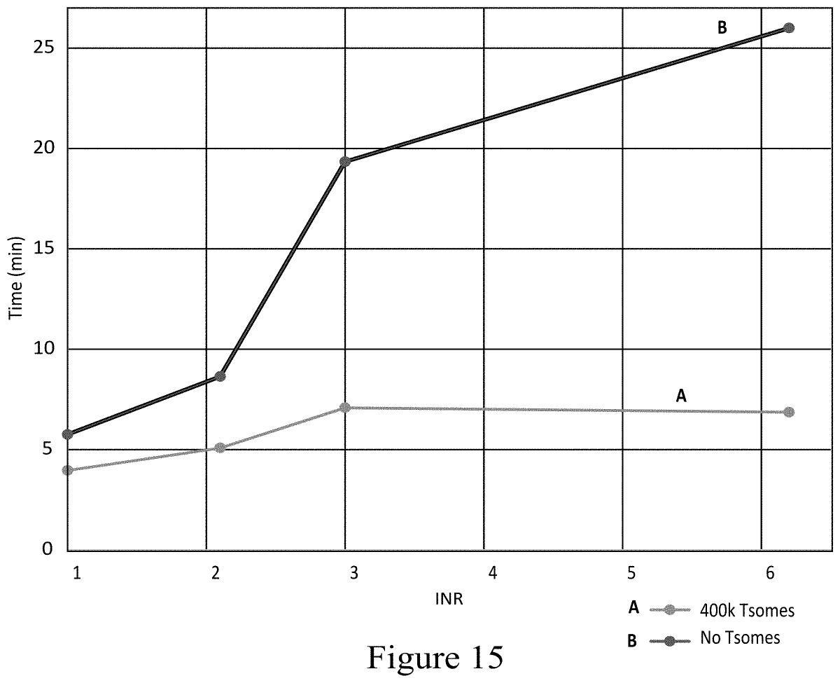

Thrombosomes decrease lag time at all tested thrombosome concentrations, and the plateau effect demonstrates no hypercoagulability (FIG. 15). Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3.

### Example 10. TEG Results

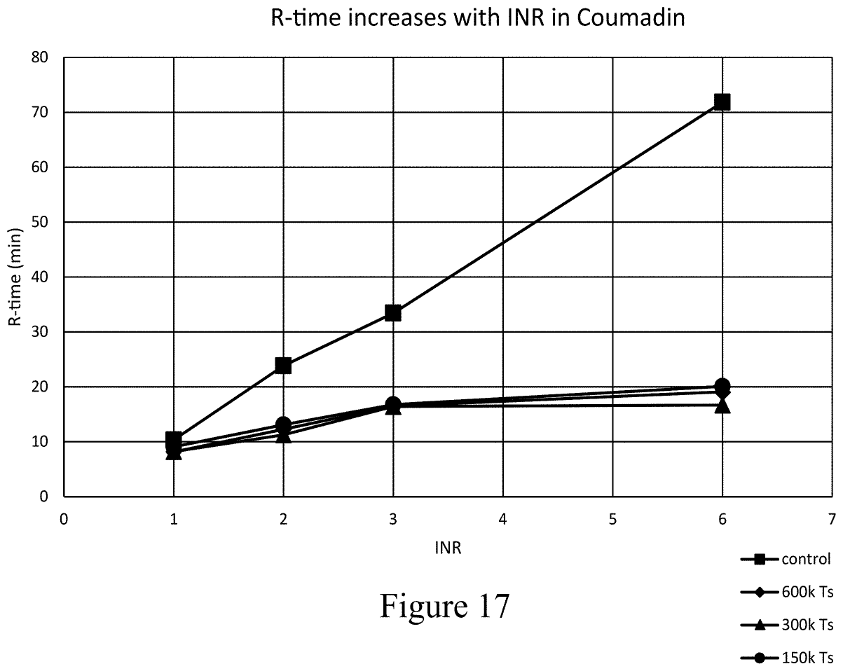

Adding various concentrations of thrombosomes decreases R-time in warfarin plasma. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. T-TAS® assays were performed according to Example 3. FIG. 17 shows that thrombosomes lower R-time for various INR values. A plateau is seen before R-times of 20 min, suggesting that thrombosomes could produce therapeutically significant results.

### Example 11. Activated Clotting Time

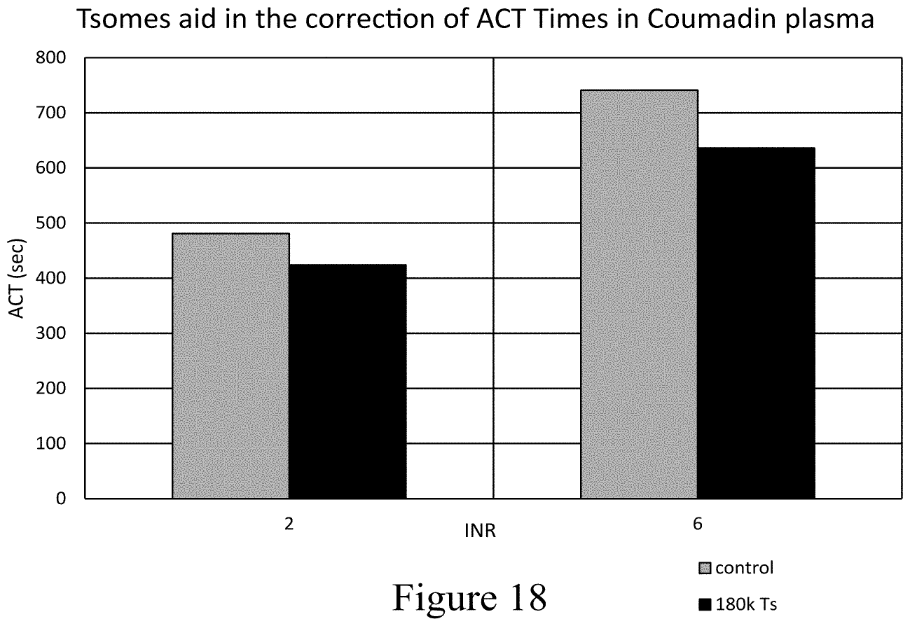

Thrombosomes exhibit an effect on activated clotting time in warfarin plasma. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. To empty MaxAct tubes, 25 μl of thrombosomes or control buffer and 25 μl of 0.2M CaCl₂) were added, followed by the addition of Whole Citrated Blood or Plasma (400 μl). The tubes were shaken once by hand then inserted into the MaxAct ACT instrument and the clotting times automatically recorded. Adding thrombosomes to physiological range improves the tACT. No change in the normal condition (INR=1) was observed. (FIG. 18).

### Example 12. Whole Blood Assays

Coumadin whole blood was prepared. Plasma from donor whole blood was removed and replaced with warfarin or control plasma as described in Example 3.

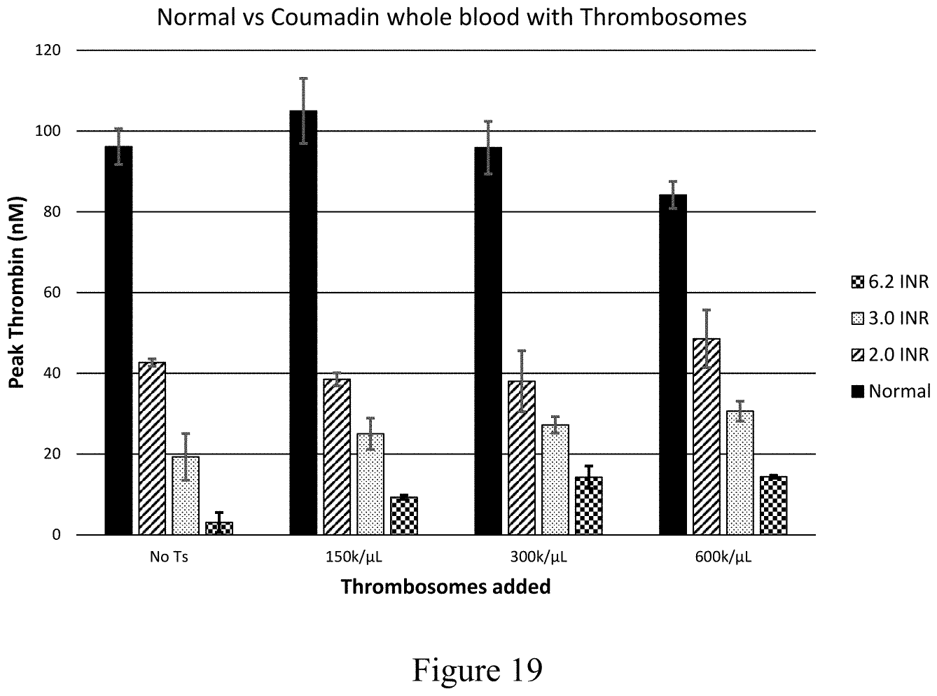

Thrombosomes increase thrombin generation in 3.0 and 6.2 INR whole blood. Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3. The thrombosomes increase peak thrombin; however, the magnitude of the effect is small. The thrombosomes exhibit minimal effect on a normal blood state (FIG. 19). In these experiments, the platelet count was 150×10³/μL (as measured by CBC of the whole blood).

### Example 13. Thrombin Generation Assays

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. TGA assays were performed as described in Example 3.

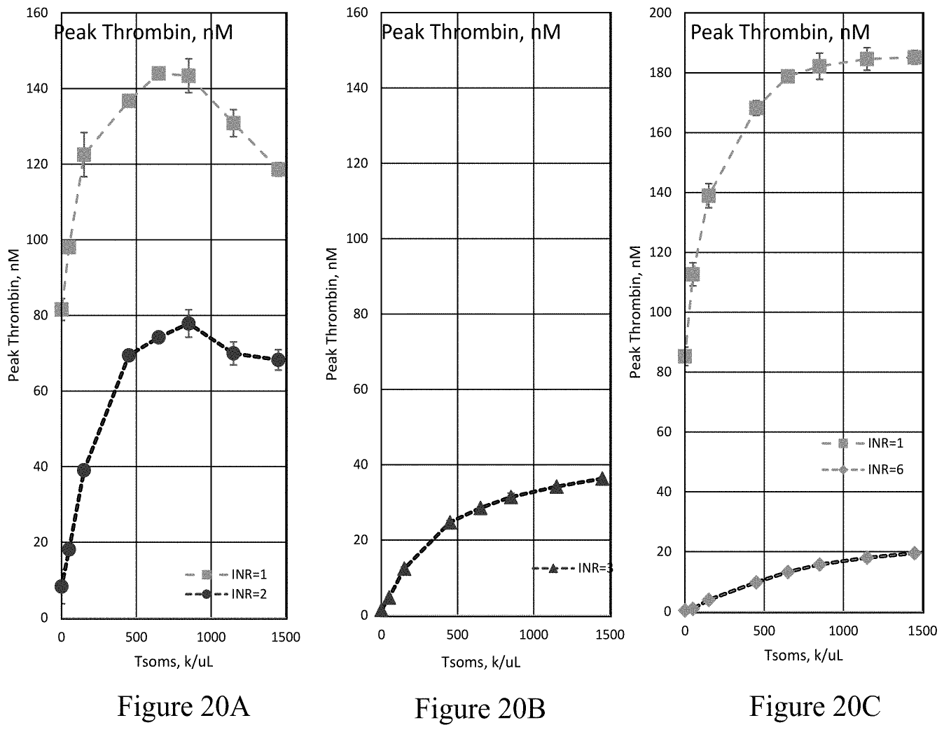

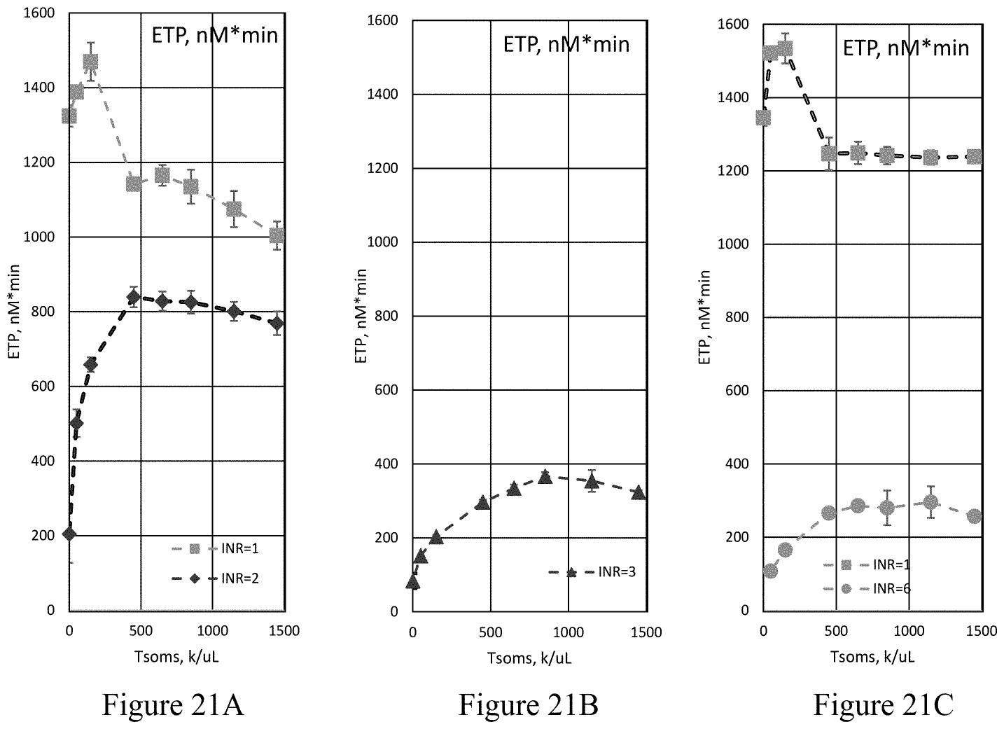

The effect of thrombosomes (batch 4) was tested and compared to standard plasma (INR=1.0) and elevated INR controls (INR=2, 3, and 6), at thrombosome concentrations of 1450, 1150, 850, 650, 450, 150, 50 and 0 k/uL. The resulting peak thrombin (FIG. 20A-C) and thrombin generation (ETP; FIG. 21A-C) values for various INR values are shown in FIGS. 20 and 21.

Peak Thrombin Results

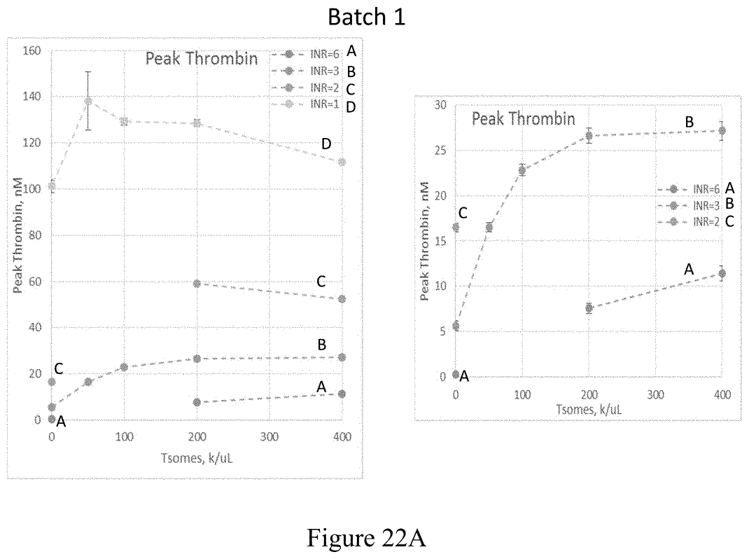

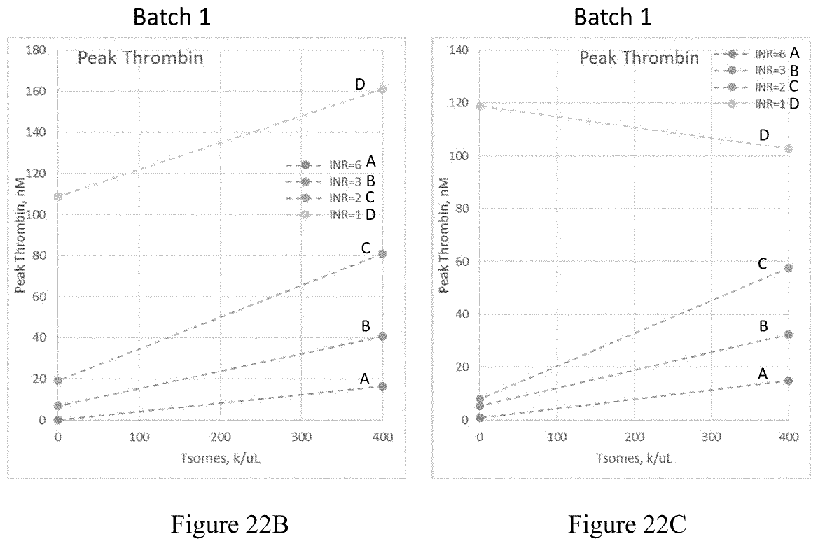

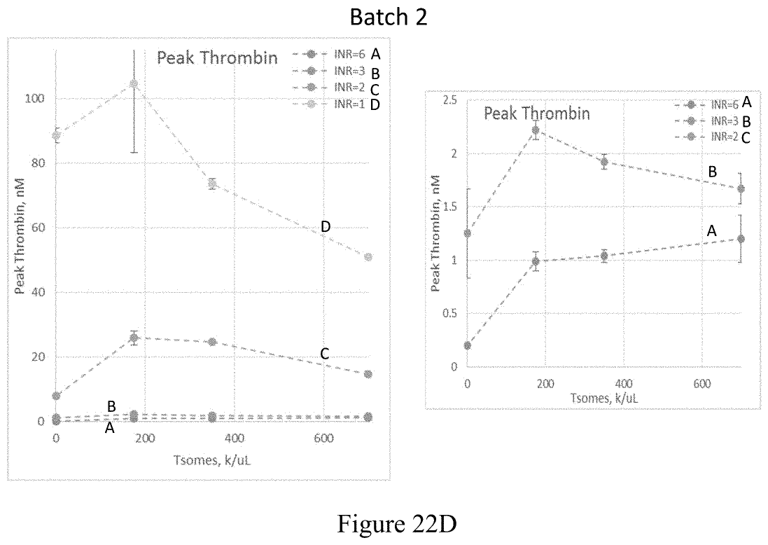

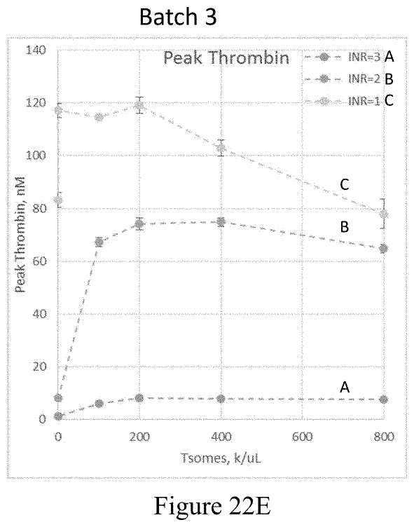

INR=1: The increase of the Peak Thrombin was saturated at about 800 k thrombosomes and was almost doubled from the normal level of about 100 nM at maximal thrombosomes concentration (FIG. 20C). Repeating the test on the same lot showed a large increase to about 145 nM at 700 k thrombosomes followed by a decrease to 120 nM at highest thrombosomes concentrations (FIG. 20A). Previous tests showed either no increase or slight increase in Peak Thrombin with following decrease at higher thrombosomes concentrations (See, e.g., FIG. 22A-E).

INR 2: Freshly prepared thrombosomes resulted in an increase of the Peak Thrombin from approximately 10 nM to about 80 nM at maximal thrombosomes concentration (FIG. 20A). Previous tests showed similar tendencies with ranges 0-20 nM to 30-80 nM (See, e.g., FIG. 22A-E).

INR 3: Freshly prepared thrombosomes resulted in an increase of the Peak Thrombin from zero to about 40 nM at maximal thrombosomes concentration (FIG. 20B). Previous tests showed similar tendencies to a maximum of about 40 nM) (batch 1; FIG. 22A-C); 1-2 nM (batch 2; FIG. 22D); 0-10 nM (batch 3; FIG. 22E).

INR 6: Freshly prepared thrombosomes resulted in an increase of the Peak Thrombin from zero to about 20 nM at maximal thrombosomes concentration (FIG. 20C). Previous tests showed similar tendencies. (See, e.g., FIG. 22A-E).

Thrombin Generation (ETP) Results

INR 1: The ETP slightly increased at 50-150 k thrombosomes and then slightly decreased to a stable level at higher thrombosomes concentrations (FIG. 17A, FIG. 17C). Previous tests showed similar tendencies (FIGS. 21A-C). ETP range was 1000-1600 nM*min.

INR 2: The ETP increased from about 200 nM*min to about 850 nM*min at highest thrombosomes concentrations (FIG. 21A). Previous tests showed similar tendencies with ranges 200-400 nM*min to 500-900 nM*min.

INR 3: The ETP value increased from about 100 nM*min to 400 nM*min at highest thrombosomes concentrations (FIG. 21B). Previous tests showed similar tendencies with range 100-350 (batch 1); 100-200 nM*min.

INR 6: The ETP value increased from about 100 nM*min to 300 nM*min at highest thrombosomes concentrations (FIG. 21C). Previous tests showed similar tendencies with the range of 100 nM*min to 200 nM*min.

### Example 14. Thrombosomes but not Fresh Platelets Restore Thrombin Generation in Heparinized Plasma

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. aPTT and thrombin generation assays were performed as described in Example 3.

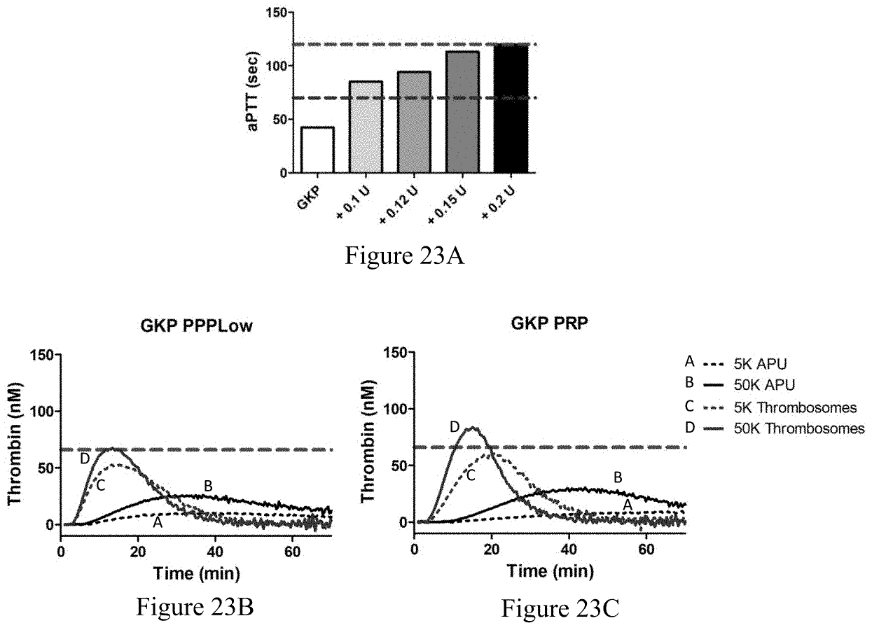

FIG. 23A shows the aPTT of George King Plasma (GKP) in the absence and presence of various concentrations of heparin as noted on the x-axis. The dashed line at approximately 70 seconds denotes the limit of abnormal aPTT and the second dashed line is the maximum time measured by the instrument (120 sec). Thrombin generation in heparin treated samples was also measured. FIG. 23B shows the effect of 0.1 U heparin in GKP on thrombin generation, in GKP, comparing apheresis units (APU) with thrombosomes at 5K (dotted lines), and 50K (solid lines) platelets or thrombosomes per μL when thrombin generation is initiated with the PPP Low reagent containing mostly phospholipids. FIG. 23C also shows thrombin generation similar to FIG. 23B, except thrombin generation is initiated by PRP reagent containing a mixture of phospholipids and tissue factor. The dashed line in FIGS. 23B and 23C denotes a typical thrombin peak value seen in this assay for control plasma. These data show that thrombosomes, but not fresh platelets, restore thrombin generation in heparinized plasma.

### Example 15. Protamine Sulfate Neutralization Restores Thrombosome-Mediated Thrombin Generation in Therapeutic Heparinized Plasma

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. aPTT and thrombin generation assays were performed as described in Example 3.

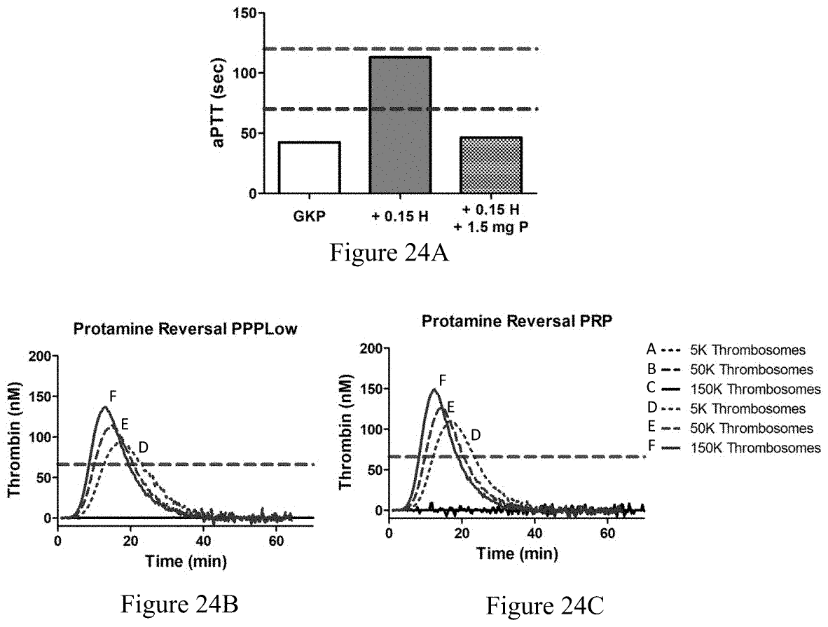

FIG. 24A shows the aPTT of George King Plasma (GKP) in the absence and presence of Heparin (H) (U/mL) and Protamine Sulfate (P) as noted on the x-axis. The dashed line at approximately 70 seconds denotes the limit of abnormal aPTT and the second dashed line is the maximum time measured by the instrument (120 sec). Thrombin generation in heparin treated samples was also measured, with and without protamine sulfate. FIG. 24B shows the effect of 2 U/mL heparin before (relatively flat lines) and after (curves) reversal by 20 μg/mL protamine sulfate on thrombin generation, in GKP, with thrombosomes at 5K (dotted line), 50K (dashed line), and 150K (solid line) thrombosomes per μL when thrombin generation is initiated with the PPP Low reagent containing mostly phospholipids. FIG. 24C also shows thrombin generation similar to FIG. 24B, except thrombin generation is initiated by PRP reagent containing a mixture of phospholipids and tissue factor. The dashed line in FIGS. 24B and 24C denotes a typical thrombin peak value seen in this assay for control plasma.

### Example 16. Thrombosomes Restore Thrombin Generation in Dabigatran-Treated Platelet Rich Plasma

Thrombosomes were prepared consistent with the procedures described in U.S. Pat. No. 8,486,617 (such as, e.g., Examples 1-5) and U.S. Pat. No. 8,097,403 (such as, e.g., Examples 1-3), and rehydrated by addition of sterile water. Thrombin generation assays were performed as described in Example 3.

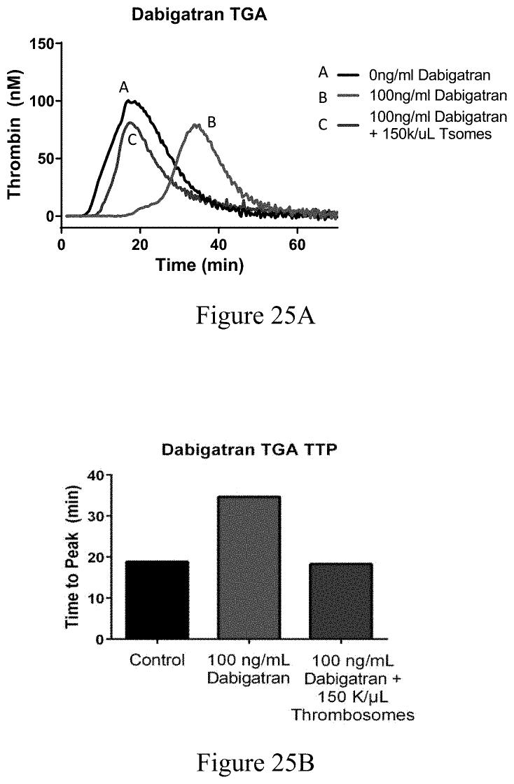

FIGS. 25A and 25B show that thrombin generation returns to normal in dabigatran treated PRP when treated with thrombosomes. Thrombin generation of PRP treated in the presence or absence of dabigatran (100 ng/mL) stimulated with PRP reagent was reversed with 150 k/μL of thrombosomes. Time to peak was increased with dabigatran to 34.67 minutes from 18.89 untreated but returned to 18.33 minutes with 150 k/μL of thrombosomes.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above. Furthermore, one having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

What is claimed is:

1. A method of treating a coagulopathy in a subject, the method comprising administering to the subject in need thereof an effective amount of a composition comprising platelets or platelet derivatives and an incubating agent comprising one or more salts, a buffer, optionally a cryoprotectant, and optionally an organic solvent.

2. A method of treating a coagulopathy in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

3. A method of restoring normal hemostasis in a subject, the method comprising administering to the subject in need thereof an effective amount of a composition comprising platelets or platelet derivatives and an incubating agent comprising one or more salts, a buffer, optionally a cryoprotectant, and optionally an organic solvent.

4. A method of restoring normal hemostasis in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

5. A method of preparing a subject for surgery, the method comprising administering to the subject in need thereof an effective amount of a composition comprising platelets or platelet derivatives and an incubating agent comprising one or more salts, a buffer, optionally a cryoprotectant, and optionally an organic solvent.

6. A method of preparing a subject for surgery, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

7. The method of any one of claims 5-6, wherein the surgery is an emergency surgery.

8. The method of any one of claims 5-6, wherein the surgery is a scheduled surgery.

9. The method of any one of claims 1-8, wherein the subject has been treated or is being treated with an anticoagulant.

10. The method of claim 9, wherein treatment with the anticoagulant is stopped.

11. The method of claim 9, wherein treatment with the anticoagulant is continued.

12. A method of ameliorating the effects of an anticoagulant in a subject, the method comprising administering to the subject in need thereof an effective amount of a composition comprising platelets or platelet derivatives and an incubating agent comprising one or more salts, a buffer, optionally a cryoprotectant, and optionally an organic solvent.

13. A method of ameliorating the effects of an anticoagulant in a subject, the method comprising administering to the subject in need thereof an effective amount of a composition prepared by a process comprising incubating platelets with an incubating agent comprising one or more salts, a buffer, optionally a cryoprotectant, and optionally an organic solvent, to form the composition.

14. The method of claim 12 or claim 13, wherein the effects of the anticoagulant are the result of an overdose of the anticoagulant.

15. The method of any one of claims 1-14, wherein the composition further comprises an anti-fibrinolytic agent.

16. The method of claim 15, wherein the anti-fibrinolytic agent is selected from the group consisting of F-aminocaproic acid (EACA), tranexamic acid, aprotinin, aminomethylbenzoic acid, fibrinogen, and a combination thereof.

17. The method of claim 15 or claim 16, wherein the platelets or platelet derivatives are loaded with the anti-fibrinolytic agent.

18. The method of any one of claims 1-17, wherein the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof.

19. The method of any one of claims 1-17, wherein the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof.

20. The method of claim 18 or claim 19, wherein the anticoagulant is warfarin.

21. The method of claim 18 or claim 19, wherein the anticoagulant is heparin.

22. The method of any one of claims 1-21, wherein before the administering, the subject had an INR of at least 4.0.

23. The method of claim 22, wherein after the administering, the subject has an INR of 3.0 or less.

24. The method of claim 22, wherein after the administering, the subject has an INR of 2.0 or less.

25. The method of any one of claims 1-21, wherein before the administering, the subject had an INR of at least 3.0.

26. The method of claim 25, wherein after the administering, the subject has an INR of 2.0 or less.

27. The method of any one of claims 1-26, wherein administering comprises administering topically.

28. The method of any one of claims 1-26, wherein administering comprises administering parenterally.

29. The method of any one of claims 1-26, wherein administering comprises administering intravenously.

30. The method of any one of claims 1-26, wherein administering comprises administering intramuscularly.

31. The method of any one of claims 1-26, wherein administering comprises administering intrathecally.

32. The method of any one of claims 1-26, wherein administering comprises administering subcutaneously.

33. The method of any one of claims 1-26, wherein administering comprises administering intraperitoneally.

34. The method of any one of claims 1-33, wherein the composition is dried prior to the administration step.

35. The method of claim 34, wherein the composition is rehydrated following the drying step.

36. The method of any one of claims 1-34, wherein the composition is freeze-dried prior to the administration step.

37. The method of claim 35, wherein the composition is rehydrated following the freeze-drying step.

38. The method of any one of claims 1-37, wherein the incubating agent comprises one or more salts selected from phosphate salts, sodium salts, potassium salts, calcium salts, magnesium salts, and a combination of two or more thereof.

39. The method of any one of claims 1-38, wherein the incubating agent comprises a carrier protein.

40. The method of any one of claims 1-39, wherein the buffer comprises HEPES, sodium bicarbonate (NaHCO₃), or a combination thereof.

41. The method of any one of claims 1-40, wherein the composition comprises one or more saccharides.

42. The method of claim 41, wherein the one or more saccharides comprise trehalose.

43. The method of claim 41 or claim 42, wherein the one or more saccharides comprise polysucrose.

44. The method of any one of claims 41-43, wherein the one or more saccharides comprise dextrose.

45. The method of any one of claims 1-44, wherein the composition comprises an organic solvent.

46. The method of any one of claims 1-45, wherein the platelets or platelet derivatives comprise thrombosomes.
